(19) United States
(12) Patent Application Publication
(10) Pub. No.: US 2026/0115307 A1
(43) Pub. Date: Apr. 30, 2026

(54) ANTI-FUCOSYL-GM1 ANTIBODY DRUG CONJUGATES

(71) Applicant: BRISTOL-MYERS SQUIBB COMPANY, Princeton, NJ (US)

(72) Inventors: Michael Wichroski, Newbury, MA (US); Nicole Vincent Jordan, Needham, MA (US); Siqi Liu, Charleston, MA (US); Brian A. Poirson, Princeton, NJ (US); Sayumi Yamazoe, Mountain View, CA (US)

(73) Assignee: BRISTOL-MYERS SQUIBB COMPANY, Princeton, NJ (US)

(21) Appl. No.: 19/373,862

(22) Filed: Oct. 30, 2025

Related U.S. Application Data
(60) Provisional application No. 63/714,460, filed on Oct. 31, 2024.

(51) Int. Cl. A61K 47/68; A61P 35/00; C07K 16/28; C07K 16/30

(52) U.S. Cl. CPC A61K 47/68037; A61K 47/6851; A61K 47/6889; A61P 35/00; C07K 16/2818; C07K 16/3084

(57) ABSTRACT

This disclosure provides combination therapy for treating a subject having a FucGM1-expressing cancer, such as a subject afflicted with lung cancer, such as small cell lung cancer, comprising administering to the subject an anti-fucosyl-GM1 antibody drug conjugate.

### CROSS-REFERENCE TO RELATED APPLICATIONS

This application claims the benefit of U.S. provisional patent application Ser. No. 63/714,460, filed Oct. 31, 2024, which is incorporated herein by reference in its entirety.

### REFERENCE TO SEQUENCE LISTING SUBMITTED ELECTRONICALLY

The content of the electronically submitted sequence listing (Name: 14613WOPCTSequenceListing.xml; Size: 12,661 bytes; and Date of Creation: Oct. 17, 2025), filed with the application, is incorporated herein by reference in its entirety.

### FIELD OF THE INVENTION

The present invention relates to an antibody-drug conjugate (ADC) comprising an antibody that specifically binds to fucosyl-GM1, a linker, and a cytotoxic moiety, and improved methods of treatment of cancer comprising administration of the ADC.

### BACKGROUND OF THE INVENTION

Fucosyl-GM1 is a sphingolipid monosialoganglioside composed of a ceramide lipid component, which anchors the molecule in the cell membrane, and a carbohydrate component that is exposed at the cell surface. Carbohydrate antigens are the most abundantly expressed antigens on the cell surface of cancers (Feizi T. (1985) Nature 314:53-7). In some tumor types, such as small cell lung cancer (SCLC), initial responses to chemotherapy are impressive, but chemo-refractory relapses rapidly follows. Intervention with novel immunotherapeutics may succeed in overcoming drug resistant relapse (Johnson D H. (1995) Lung Cancer 12 Suppl 3:S71-5). Several carbohydrate antigens, such as gangliosides GD3 and GD2, have been shown to function as effective targets for passive immunotherapy with mAbs (Irie R F and Morton D L. (1986) PNAS 83:8694-8698; Houghton A N et al. (1985) PNAS 82:1242-1246). Ganglioside antigens have also been demonstrated to be effective targets for active immunotherapy with vaccines in clinical trials (Krug L M et al. (2004) Clinical Cancer Research 10:6094-6100; Dickler M N et al. (1999) Clinical Cancer Research 5:2773-2779; Livingston P O et al. (1994) J. Clin. Oncol. 12:1036-44). Indeed, serum derived from SCLC patients who developed antibody titers to fucosyl-GM1 following vaccination with KLH conjugated antigen, demonstrated specific binding to tumor cells and tumor specific complement dependent cytotoxicity (CDC). Anti-fucosyl-GM1 titer associated toxicities were mild and transient and three patients with limited-stage SCLC were relapse-free at 18, 24, and 30 months (Krug et al., supra; Dickler et al., supra).

Fucosyl-GM1 expression has been shown in a high percentage of SCLC cases while having little or no expression in normal tissues (Nilsson et al. (1984) Glycoconjugate J. 1:43-9; Krug et al., supra; Brezicka et al. (1989) Cancer Res. 49:1300-5; Zhangyi et al. (1997) Int. J Cancer 73:42-49; Brezicka et al. (2000) Lung Cancer 28:29-36; Fredman et al. (1986) Biochim. Biophys. Acta 875: 316-23; Brezicka et al. (1991) APMIS 99:797-802; Nilsson et al. (1986) Cancer Res. 46:1403-7). The presence of fucosyl-GM1 has been demonstrated in culture media from SCLC cell lines, in tumor extracts and serum of nude mouse xenografts and in the serum of SCLC patients with extensive-stage disease (Vangsted et al. (1991) Cancer Res. 51:2879-84; Vangsted et al. (1994) Cancer Detect. Prev. 18:221-9). Besides SCLC, fucosyl-GM1 expression has also been observed in a significant fraction of non-small cell lung cancer (NSCLC) samples (WO 07/067992). These reports provide convincing evidence for fucosyl-GM1 as a highly specific tumor antigen, which may be targeted by an immunotherapeutic agent.

An antibody that recognizes fucosyl-GM1 on cancer cells and directs their destruction, anti-fucosyl-GM1 mAb BMS-986012, has entered clinical trials for the treatment of subjects with relapsed/refractory small cell lung cancer (NCT02247349). See Molckovsky & Siu (2008) J. Hematol. Oncol. 1:20. BMS-986012 is a non-fucosylated antibody and thus exhibits enhanced antibody-dependent cytotoxicity (ADCC) through increased binding to CD16 expressed on effector immune cells. Although effective in the pre-clinical settings, BMS-986012 did not demonstrate adequate monotherapy efficacy in patients (Chu et al., 2022 JTO Clin Res Rep 3:100400). Therefore, there exists a need for more effective cancer therapy based on targeting of fucosyl-GM1-expressing cancer cells and killing of those cells using a cytotoxic agent.

An antibody-drug conjugate (ADC) is a modality that has emerged as an important class of molecules in the treatment of solid cancers. Trastuzumab deruxtecan demonstrated compelling efficacy in patients with HER2 positive breast cancers (Cortes et al., 2022 N Engl J Med 386:1143-1154). An ADC may be used to deliver cytotoxic payloads, e.g., the topoisomerase-I inhibitor (TOPO-I) family (Okamoto et al., 2020 Xenobiotica 50:1242-1250). In small cell lung cancer (SCLC), however, there are no approved ADCs. Thus, there is a need in the field for improved treatments for solid cancers such as SCLC. Since SCLC is sensitive to TOPO-I as a small molecule chemotherapy agent (O'Brien et al., 2006 J Clin Oncol 24:5441-5447), Applicants have here developed improved cancer treatments by pairing a fucosyl-GM1 targeting antibody with a TOPO-I payload to form ADCS that may provide exceptional therapeutic benefit.

### SUMMARY OF THE INVENTION

The present disclosure provides an antibody drug conjugate (ADC) having the formula (I),

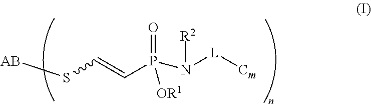

or a pharmaceutically acceptable salt, a stereoisomer or a solvate thereof,
wherein:
[symbol] indicates that the configuration of the double bond may be E or Z;
R¹ is a polyalkene glycol unit comprising at least 3 alkylene glycol subunits;
R² is H, or an optionally substituted aliphatic residue, or an optionally substituted aromatic residue;
L is a linker;
C is a cytotoxic moiety;
m is an integer ranging from 1 to 10;
n ranges from 1 to 20; and
AB is an anti-fucosyl-GM1 antibody, or an antigen-binding portion thereof.

In certain embodiments, the AB is the anti-fucosyl-GM1 antibody.

In certain embodiments, the anti-fucosyl-GM1 antibody, or an antigen-binding portion thereof comprises a heavy chain variable region (VH) and a light chain variable region (VL), wherein the VH comprises complementarity determining region (CDR) 1 (CDRH1) comprising the amino acid sequence as set forth in SEQ ID NO: 5, CDRH2 comprising the amino acid sequence as set forth in SEQ ID NO: 6, and CDRH3 comprising the amino acid sequence as set forth in SEQ ID NO: 7 and the VL comprises CDRL1 comprising the amino acid sequence as set forth in SEQ ID NO: 8, CDRL2 comprising the amino acid sequence as set forth in SEQ ID NO: 9, and CDRL3 comprising the amino acid sequence as set forth in SEQ ID NO: 10.

In certain embodiments, the ADC binds to human cells expressing fucosyl-GM1.

In certain embodiments, the ADC is internalized into human cells expressing fucosyl-GM1.

In certain embodiments of the ADC, the VH comprises the amino acid sequence set forth in SEQ ID NO: 1 and the VL comprises the amino acid sequence set forth in SEQ ID NO: 2.

In certain embodiments of the ADC, the anti-fucosyl-GM1 antibody, or antigen-binding portion thereof, comprises a heavy chain variable region comprising:
i. CDRH1 comprising the sequence set forth in SEQ ID NO: 5;
ii. CDRH2 comprising the sequence set forth in SEQ ID NO: 6; and
iii. CDRH3 comprising the sequence set forth in SEQ ID NO: 7.

In certain embodiments of the ADC, the anti-fucosyl-GM1 antibody, or antigen-binding portion thereof, comprises a light chain variable region comprising:
i. CDRL1 comprising the sequence set forth in SEQ ID NO: 8;
ii. CDRL2 comprising the sequence set forth in SEQ ID NO: 9; and
iii. CDRL3 comprising the sequence set forth in SEQ ID NO: 10.

In certain embodiments of the ADC, the anti-fucosyl-GM1 antibody, or antigen-binding portion thereof, comprises:
a. a heavy chain variable region comprising:
   i. CDRH1 comprising the sequence set forth in SEQ ID NO: 5;
   ii. CDRH2 comprising the sequence set forth in SEQ ID NO: 6; and
   iii. CDRH3 comprising the sequence set forth in SEQ ID NO: 7; and;
b. a light chain variable region comprising:
   i. CDRL1 comprising the sequence set forth in SEQ ID NO: 8;
   ii. CDRL2 comprising the sequence set forth in SEQ ID NO: 9; and
   iii. CDRL3 comprising the sequence set forth in SEQ ID NO: 10.

In certain embodiments of the ADC, the heavy chain variable region comprises the sequence set forth in SEQ ID NO: 1.

In certain embodiments of the ADC, the anti-fucosyl-GM1 antibody, or antigen-binding portion thereof, comprises a heavy chain variable region that is at least 80%-85%, at least 85%-90%, at least 90%-95%, or about 95%-99% identical to the amino acid sequence set forth in SEQ ID NO: 1. In certain embodiments of the ADC, the anti-fucosyl-GM1 antibody, or antigen-binding portion thereof, comprises a heavy chain variable region that is at least about 80%, at least about 85%, at least about 90%, at least about 95%, at least about 96%, at least about 97%, at least about 98%, or at least about 99% identical to the amino acid sequence set forth in SEQ ID NO: 1. In certain embodiments of the ADC, the anti-fucosyl-GM1 antibody, or antigen-binding portion thereof, comprises a heavy chain variable region that is at least 80%, at least 85%, at least 90%, at least 95%, at least 96%, at least 97%, at least 98%, or at least 99% identical to the amino acid sequence set forth in SEQ ID NO: 1.

In certain embodiments of the ADC, the light chain variable region comprising the sequence set forth in SEQ ID NO: 2.

In certain embodiments of the ADC, the anti-fucosyl-GM1 antibody, or antigen-binding portion thereof, comprises a light chain variable region that is at least 80%-85%, at least 85%-90%, at least 90%-95%, or about 95%-99% identical to the amino acid sequence set forth in SEQ ID NO: 2. In certain embodiments of the ADC, the anti-fucosyl-GM1 antibody, or antigen-binding portion thereof, comprises a light chain variable region that is at least about 80%, at least about 85%, at least about 90%, at least about 95%, at least about 96%, at least about 97%, at least about 98%, or at least about 99% identical to the amino acid sequence set forth in SEQ ID NO: 2. In certain embodiments of the ADC, the anti-fucosyl-GM1 antibody, or antigen-binding portion thereof, comprises a light chain variable region that is at least 80%, at least 85%, at least 90%, at least 95%, at least 96%, at least 97%, at least 98%, or at least 99% identical to the amino acid sequence set forth in SEQ ID NO: 2.

In certain embodiments of the ADC, the heavy chain variable region comprises the amino acid sequence set forth in SEQ ID NO: 1; and the light chain variable region comprises the amino acid sequence set forth in SEQ ID NO: 2.

In certain embodiments of the ADC, the anti-fucosyl-GM1 antibody, or antigen binding portion thereof, comprises a heavy chain comprising the amino acid sequence set forth in SEQ ID NO: 3 or SEQ ID NO: 11.

In certain embodiments of the ADC, the anti-fucosyl-GM1 antibody, or antigen-binding portion thereof, comprises a light chain comprising the amino acid sequence set forth in SEQ ID NO: 4 or SEQ ID NO: 12.

In certain embodiments of the ADC, the anti-fucosyl-GM1 antibody, or antigen-binding portion thereof, comprises a heavy chain that is at least 80-99% identical to the amino acid sequence set forth in SEQ ID NO: 3 or SEQ ID NO:11. In certain embodiments of the ADC, the anti-fucosyl-GM1 antibody, or antigen-binding portion thereof, comprises a light chain that is at least 80%-85%, at least 85%-90%, at least 90%-95%, or at least 95%-99% identical to the amino acid sequence set forth in SEQ ID NO:3. In certain embodiments of the ADC, the anti-fucosyl-GM1 antibody, or antigen-binding portion thereof, comprises a light chain that is at least 80%-85%, at least 85%-90%, at least 90%-95%, or about 95%-99% identical to the amino acid sequence set forth in SEQ ID NO: 11.

In certain embodiments of the ADC, the anti-fucosyl-GM1 antibody, or antigen-binding portion thereof, comprises a light chain that is at least 80-99% identical to the amino acid sequence set forth in SEQ ID NO: 4 or SEQ ID NO: 12. In certain embodiments of the ADC, the anti-fucosyl-GM1 antibody, or antigen-binding portion thereof, comprises a light chain that is at least 80%-85%, at least 85%-90%, at least 90%-95%, or at least 95%-99% identical to the amino acid sequence set forth in SEQ ID NO: 4. In certain embodiments of the ADC, the anti-fucosyl-GM1 antibody, or antigen-binding portion thereof, comprises a light chain that is at least 80%-85%, at least 85%-90%, at least 90%-95%, or at least 95%-99% identical to the amino acid sequence set forth in SEQ ID NO: 12.

In certain embodiments of the ADC, wherein the anti-fucosyl-GM1 antibody, or antigen-binding portion thereof, comprises: a heavy chain comprising the sequence of SEQ ID NO: 3; and a light chain comprising the sequence of SEQ ID NO: 4. In various embodiments, the ADC comprises the anti-fucosyl-GM1 antibody. In certain embodiments of the ADC, the anti-fucosyl-GM1 antibody comprises: a heavy chain comprising the sequence of SEQ ID NO: 3; and a light chain comprising the sequence of SEQ ID NO: 4.

In certain embodiments of the ADC, the anti-fucosyl-GM1 antibody, or antigen-binding portion thereof, comprises: a heavy chain comprising the amino acid sequence set forth in SEQ ID NO: 11; and a light chain comprising the amino acid sequence set forth in SEQ ID NO: 12. In various embodiments, the ADC comprises the anti-fucosyl-GM1 antibody comprising a heavy chain comprising the amino acid sequence set forth in SEQ ID NO: 11; and a light chain comprising the amino acid sequence set forth in SEQ ID NO: 12.

In certain embodiments of the ADC, the anti-fucosyl-GM1 antibody is non-fucosylated.

In certain embodiments of the ADC, the anti-fucosyl-GM1 antibody comprises an IgG1 constant domain.

In certain embodiments of the ADC, having the formula (II):

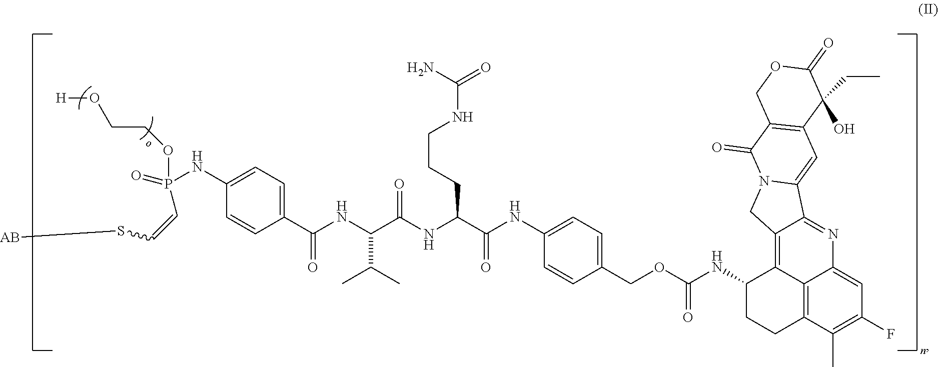

or a pharmaceutically acceptable salt, a stereoisomer or a solvate thereof, wherein
n ranges from about 4 to 9;
o is an integer ranging from 10 to 30; and
AB is an anti-FucGM1 antibody or antigen binding portion thereof comprising a VH and a VL, which VH comprises the amino acid sequence set forth in SEQ ID NO: 1, and the VL comprise the amino acid sequence set forth in SEQ ID NO: 2, respectively.

In certain embodiments, the AB is the anti-fucosyl-GM1 antibody.

In certain embodiment of the ADCs, the anti-fucosyl-GM1 antibody, or antigen-binding portion thereof, comprises a heavy chain comprising the sequence of SEQ ID NO: 3.

In certain embodiments of the ADCs, the anti-fucosyl-GM1 antibody, or antigen-binding portion thereof, comprises a light chain comprising the sequence of SEQ ID NO: 4.

In certain embodiments of the ADCs, the anti-fucosyl-GM1 antibody, or antigen-binding portion thereof, comprises a heavy chain comprising the sequence of SEQ ID NO: 3; and a light chain comprising the sequence of SEQ ID NO: 4.

In certain embodiments of the ADCs, the anti-fucosyl-GM1 antibody, or antigen-binding portion thereof, comprises a heavy chain comprising the sequence of SEQ ID NO: 11.

In certain embodiments of the ADCs, the anti-fucosyl-GM1 antibody, or antigen-binding portion thereof, comprises a light chain comprising the sequence of SEQ ID NO: 12.

In certain embodiments of the ADCs, the anti-fucosyl-GM1 antibody, or antigen-binding portion thereof, comprises a heavy chain comprising the sequence of SEQ ID NO: 11; and a light chain comprising the sequence of SEQ ID NO: 12. In certain embodiments of the ADCs, the ADC comprises the anti-fucosyl-GM1 antibody comprising a heavy chain comprising the sequence of SEQ ID NO: 11; and a light chain comprising the sequence of SEQ ID NO: 12.

In certain embodiments of the ADCs, the n is 4, 5, 6, 7, 8, or 9. In certain embodiments of the ADCs, the n is 4. In certain embodiments of the ADCs, the n is 5. In certain embodiments of the ADCs, the n is 6. In certain embodiments of the ADCs, the n is 7. In certain embodiments of the ADCs, the n is 8. In certain embodiments of the ADCs, the n is 9.

In certain embodiments of the ADCs, the n is about 4, about 5, about 6, about 7, about 8, or about 9.

In certain embodiments of the ADCs, the o is an integer ranging from 8 to 16.

In certain embodiments of the ADCs, the o is 10, 11, 12, 13 or 14.

In certain embodiments of the ADCs, the o is an integer ranging from 20 to 28 In certain embodiments of the ADCs, the o is 22, 23, 24, 25, or 26.

In certain embodiments of the ADCs, the n is an integer ranging from 6 to 8.

In certain embodiments of the ADCs, the o is 24 and n is 4.

In certain embodiments of the ADCs, the o is 24 and n is 8.

In certain embodiments of the ADCs, the antibody is a human, humanized, or chimeric antibody.

In certain embodiments of the ADC, the antigen binding portion thereof comprises a Fab, Fab′, (Fab′)2, Fv, or scFv fragment.

The present disclosure provides a kit comprising any ADC described herein and instructions for use.

The present disclosure provides an in vitro method of killing cells expressing FucGM1 comprising contacting a sample comprising the cells with any ADC described herein. For example, the ADC is ADC-001 or ADC-002. In certain embodiments, the ADC is ADC-001. In certain embodiments, the ADC is ADC-002. In certain embodiments, the sample is a tumor sample comprising cancer cells.

The present disclosure provides a method of treating cancer associated with expression of FucGM1 comprising administering to a subject in need thereof any ADC described herein. In certain embodiments, the ADC is ADC-001. In certain embodiments, the ADC is ADC-002.

In certain embodiments of the method, the cancer comprises colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, myelodysplastic syndromes, or any combination thereof. In certain embodiments of the method, the lung cancer is SCLC.

In certain embodiments, the method further comprises administering one or more additional therapies. In certain embodiments of the method, the one or more additional therapies comprises radiation therapy, chemotherapy, immune checkpoint inhibitor therapy, CAR-T therapy, immunosuppressive therapy, immunostimulatory therapy, cell therapy, a therapeutic agent, or any combination thereof.

In certain embodiments of the method, the immune checkpoint therapy comprises administering an immune checkpoint inhibitor. In certain embodiments of the method, the immune checkpoint inhibitor comprises an anti-PD-1 antibody, an anti-PD-L1 antibody, an anti-LAG-3 antibody, an anti-CTLA-4 antibody, an anti-TIGIT antibody, an anti-TIM3 antibody, or any combination thereof. In certain embodiments of the method, the immune checkpoint inhibitor is the anti-PD1 antibody.

An aspect of the disclosure provides a method of producing any of the ADCs described herein comprising conjugating an anti-FucGM1 antibody, or antigen binding portion thereof described herein (e.g., Table 3), with a linker and a cytotoxic moiety under suitable conditions. In certain embodiments, the method comprises preparing an ADC of the formula (I), comprising reacting a compound of the formula (III):

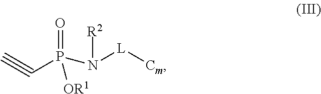

with a thiol containing compound, AB-(SH)n, wherein AB is the anti-FucGM1 antibody, or antigen binding portion thereof, to yield the ADC of the formula (I), R¹, R², L, C, m, and n being as defined in the formula (I).

In certain embodiments, the process is described herein in the Working Examples, for example, Working Example 2 or Working Example 6. In certain embodiments, the anti-FucGM1 antibody is MBN-001. In certain embodiments, the compound of formula (III) is Compound A′. In certain embodiments, the ADC produced by the method is ADC-002.

### BRIEF DESCRIPTION OF THE DRAWINGS

FIG. 1A and FIG. 1B are a set of representative sensograms (binding response versus time) showing different stages of binding between biotinylated FucGM1 (FIG. 1A) or biotinylated-GM1 (FIG. 1B) and anti-FucGM1 antibody MBN-001 and the ADC-002 (DAR8) ADC.

FIGS. 2A-2D are graphs showing the percent inhibition across a panel of FucGM1-expressing cell lines. The graphs show percent (%) growth inhibition by MBN-001, ADC-001, isotype control conjugated to Dxd, or Dxd of cell lines: DMS-79 (FIG. 2A), NCI-H187 (FIG. 2B), DMS-53 (FIG. 2C), and Raji (FIG. 2D). IC50 values for ADC-001 are listed on the graphs.

FIGS. 3A-3D are a series of graphs showing the in vitro characterization of MBN-001 compared to ADC-002 showing cellular binding (FIG. 3A), internalization (FIG. 3B, left), ADCC activity (FIG. 3C, left and right), and ADC cytotoxicity (FIG. 3D). FIG. 3B (right) also includes an image to demonstrate lysosomal co-localization with lysotracker for the ADC-002 treated cells.

FIGS. 4A-4C are graphs showing in vivo efficacy of ADC-001 and ADC-002.

Both ADC-001 and ADC-002 showed dose-dependent efficacy in DMS-79 in vivo tumor model, leading to complete tumor regression at 3 mg/kg (3 mpk) dose after a single intravenous injection. (FIG. 4A). ADC-001 had superior efficacy in vivo compared to MBN-001 (ADCC only) or ADCN001 (inert Fc; ADC only) (FIG. 4B). ADC-002 had superior efficacy in vivo compared to MBN-001 (ADCC only) or ADCN002 (inert Fc; ADC only) (FIG. 4C).

FIG. 5A and FIG. 5B are graphs showing ADC-002's dual ADC and ADCC MOA works in conjunction in vivo. ADC-002 induced DNA damage response biomarker phospho-CHK1 (pCHK1) and phospho-yH2Ax (p-gH2Ax) in tumor cells in vivo, and the response peaked on day 3 (FIG. 5A, left and right). ADC-002 induced NK and macrophage infiltration into the tumor tissue, and the response peaked on day 6 (FIG. 5B, left and right).

FIGS. 6A-6C are graphs showing the comparison between fucosyl-GM1 and B7H3 as ADC targets for SCLC. Absolute receptor density quantification of fucosyl-GM1 and B7H3 in DMS-79 cells were quantified (FIG. 6A). In vitro cytotoxicity comparison between ADC-002 and B7H3-DXd was also compared (FIG. 6B). In vivo efficacy comparison between ADC-002 and B7H3-DXd in the DMS-79 model after a single intravenous injection with either 0.3 mg/kg (mpk) or 1 mpk of the ADCs was also determined (FIG. 6C).

### DETAILED DESCRIPTION OF THE INVENTION

BMS-986012 is a fully human monoclonal antibody (mAb) that specifically binds to the fucosyl-GM1 ganglioside. BMS-986012 exhibits high-affinity and dose-dependent saturable binding to fucosyl-GM1 and shows no detectable antigen-specific binding to closely related molecule GM1. Because fucosyl-GM1 is preferentially found on the surface of lung cancer cells, BMS-986012 is particularly well suited to treating lung cancer, such as SCLC.

BMS-986012 is non-fucosylated (lacking fucosylation on the Fc domain) monoclonal antibody. The absence of the fucosyl group in BMS-986012 confers higher affinity for Fc receptors resulting in enhanced antibody-dependent cellular cytotoxicity (ADCC). See, e.g., WO 2007/067992, the content of which is expressly incorporated herein by reference in its entirety.

Although BMS-986012 is in clinical trials and has been shown to be an effective monotherapy in treatment of lung cancer, improved methods of treatment are desired.

The present disclosure provides an ADC, a pharmaceutical composition comprising the ADC, and the use of the ADC in a method of treating disease, such as cancer, e.g., SCLC. Furthermore, the ADC was shown to mediate ADCC, to produce potent complement dependent cytotoxicity (CDC) as well as antibody-dependent cellular phagocytosis (ADCP).

Various aspects of the invention are described in further detail in the following subsections.

### I. Definitions

In order for the following detailed description to be readily understood, certain terms are first defined. Additional definitions are provided throughout.

The term “alkyl” by itself or as part of another term in general refers to a substituted or unsubstituted straight chain or branched, saturated hydrocarbon having the indicated number of carbon atoms; e.g., “—(C₁-C₈)alkyl” or “—(C₁-C₁₀)alkyl” refer to an alkyl group having from 1 to 8 or 1 to 10 carbon atoms, respectively. When the number of carbon atoms is not indicated, the alkyl group may have from 1 to 8 carbon atoms. Representative straight chain —(C₁-C₈)alkyl groups include, but are not limited to, -methyl, -ethyl, -n-propyl, -n-butyl, -n-pentyl, -n-hexyl, -n-heptyl and -n-octyl; branched-(C₁-C₈)alkyl groups include, but are not limited to, -isopropyl, -sec-butyl, -isobutyl, -tert-butyl, -isopentyl, and -2-methylbutyl. In some aspects, an alkyl group may be unsubstituted. Optionally, an alkyl group may be substituted, e.g., with one or more groups.

The terms “substituted”, “optionally substituted”, “optionally may be substituted” or the like, unless otherwise indicated, in general means that one or more hydrogen atoms can be each independently replaced with a substituent. Typical substituents include, but are not limited to, —X, —R, —O⁻, —OR, —SR, —S—, —NR₂, ═NR, —CX₃, —CN, —OCN, —SCN, —N═C═O, —NCS, —NO, —NO₂, ═N₂, —N₃, —NRC(═O)R, —C(═O)R, —C(═O)NR₂, —SO₃—, —SO₃H, —S(═O)₂R, —OS(═O)₂OR, —S(═O)₂NR, —S(═O)R, —OP(═O)(OR)₂ —P(═O)(OR)₂, —PO₄³⁻, —PO₃H₂, —C(═O)R, —C(═O)X, —C(═S)R, —CO₂R, —CO₂H, —C(═S)OR, —C(═O)SR, —C(═S)SR, —C(═O)NR₂, —C(═S)NR₂, or —C(═NR)NR₂, where each X is independently a halogen: —F, —Cl, —Br, or —I; and each R is independently —H, —(C₁-C₂₀)alkyl (such as e.g. —(C₁-C₁₀)alkyl or —(C₁-C₈)alkyl), —(C₆-C₂₀)aryl, (such as e.g. —(C₆-C₁₀)aryl or, e.g., —C₆-aryl), —(C₃-C₁₄)heterocycle (such as e.g. —(C₃-C₁₀)heterocycle or —(C₃-C₈)heterocycle), a protecting group, or a prodrug moiety. Typical substituents also include (═O).

The term “aliphatic or aromatic residue”, as used herein, in general refers to an aliphatic substituent, such as e.g., but not limited to an alkyl residue, which, however, can be optionally substituted by further aliphatic and/or aromatic substituents. As non-limiting examples an aliphatic residue can be a nucleic acid, an enzyme, a co-enzyme, a nucleotide, an oligonucleotide, a monosaccharide, a polysaccharide, a polymer, a fluorophore, optionally substituted benzene, etc., as long as the direct link of such a molecule to the core structure (in case of R′, e.g., the link to the nitrogen atom of the Y) is aliphatic. An aromatic residue is a substituent, wherein the direct link to the core structure is part of an aromatic system, e.g., an optionally substituted phenyl or triazolyl or pyridyl or nucleotide, as non-limiting example if the direct link of the nucleotide to the core structure is for example via a phenyl-residue. The term “aromatic residue”, as used herein, also includes a heteroaromatic residue.

The term “antibody” as used herein includes whole antibodies and any antigen binding portions (i.e., “antigen-binding portions”) or single chains thereof. An “antibody” refers, in one aspect, to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. In certain naturally occurring antibodies, the heavy chain constant region is comprised of three domains, CH1, CH2, and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁵ to 10¹¹ M or less. Any K_{D} greater than about 10⁴ M is generally considered to indicate nonspecific binding. As used herein, an antibody that “binds specifically” to an antigen refers to an antibody that binds to the antigen and substantially identical antigens with high affinity, which means having a K_{D} of 10⁷ M or less, 10⁸ M or less, 1×10⁹ M or less, 1×10¹⁰ M or less, or 1×10¹¹ M or less. In some aspects, the antibody specifically binds to an antigen with a K_{D} between 10⁸ M and 10¹⁰ M or between 10⁹ M and 10¹¹ M, but does not bind with high affinity to unrelated antigens.

An “antibody” according to the present disclosure includes, but is not limited to, naturally and non-naturally occurring antibodies, monoclonal antibodies, polyclonal antibodies, chimeric antibodies, humanized antibodies, human antibodies, nonhuman antibodies, bivalent antibodies, bispecific antibodies, multispecific antibodies, single chain antibodies, diabodies, and nanobodies.

An “isolated antibody,” as used herein, refers to an antibody which is substantially free of other antibodies having different antigenic specificities. The phrase “antigen binding portion” of an antibody, as used herein, refers to one or more fragments of an antibody that retain the ability to specifically bind to an antigen (e.g., human FucGM1). It has been shown that the antigen-binding function of an antibody can be performed by fragments of a full-length antibody. Examples of binding fragments of an antibody include (i) a Fab fragment, a monovalent fragment consisting of the V_{L}, V_{H}, CL and CH1 domains; (ii) a F(ab′)2 fragment, a bivalent fragment comprising two Fab fragments linked by a disulfide bridge at the hinge region; (iii) an Fd fragment consisting of the V_{H} and CH1 domains; (iv) an Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Antigen-binding portions can be produced by recombinant DNA techniques, or by enzymatic or chemical cleavage of intact immunoglobulins.

Antibody fragments within the scope of the present invention also include F(ab′)2 fragments which may be produced by enzymatic cleavage of an IgG by, for example, pepsin. Fab fragments may be produced by, for example, reduction of F(ab′)2 with dithiothreitol or mercaptoethylamine. A Fab fragment is a VL-CL chain appended to a VH-CH1 chain by a disulfide bridge. A F(ab′)2 fragment is two Fab fragments which, in turn, are appended by two disulfide bridges. The Fab portion of an F(ab′)2 molecule includes a portion of the Fc region between which disulfide bridges are located.

As used herein, “isotype” refers to the antibody class (e.g., IgG (including IgG1, IgG2, IgG3, and IgG4), IgM, IgA (including IgA1 and IgA2), IgD, and IgE antibody) that is encoded by the heavy chain constant region genes of the antibody.

An antibody may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids.

As used herein, the term “allotype” refers to naturally occurring variants within a specific isotype group, where the variants differ in a few amino acids. Anti-FucGM1 antibodies described herein can be of any allotype.

As used herein, the term “hypervariable region” (sometimes referred to as the “variable region”) refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35B (CDRH1), 50-65 (CDRH2) and 95-102 (CDRH3) in the heavy chain variable domain; Kabat et al., (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, (1987) J. Mol. Biol. 196: 901-917).

As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing. Amino acid residues in antibodies can also be defined using other numbering systems, such as Chothia, enhanced Chothia, IMGT, Kabat/Chothia composite, Honegger (AHo), Contact, or any other conventional antibody numbering scheme.

Chothia refers to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop. This variation is, in part, because the Kabat numbering scheme places the insertions at H35A and H35B; if neither 35A nor 35B is present, the loop ends at 32; if only 35A is present, the loop ends at 33; if both 35A and 35B are present, the loop ends at 34).

CDR region sequences may also be defined by the AbM, Contact, and IMGT naming system. The AbM hypervariable regions represent a compromise between the Kabat CDRs and Chothia structural loops, and are used by Oxford Molecular's AbM antibody modeling software (see, e.g., Antibody Engineering Vol. 2 (Kontermann and D0bel eds., 2d ed. 2010)). The_“contact” hypervariable regions are based on an analysis of the available complex crystal structures. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art.

Recently, a universal numbering system has been developed and widely adopted, ImMunoGeneTics (IMGT) Information System® (Lafranc et al., 2003, Dev. Comp. Immunol. 27(1):55-77). IMGT is an integrated information system specializing in immunoglobulins (IG), T cell receptors (TCR), and major histocompatibility complex (MHC) of human and other vertebrates. Herein, the CDRs are referred to in terms of both the amino acid sequence and the location within the light or heavy chain.

An additional numbering system (AHon) has been developed by Honegger and Plückthun, 2001, J. Mol. Biol. 309: 657-70.

As the “location” of the CDRs within the structure of the immunoglobulin variable domain is conserved between species and present in structures called loops, by using numbering systems that align variable domain sequences according to structural features, CDR and framework residues are readily identified. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are noted in the Table 1 below.

TABLE 1
CDR locations in different naming systems

| | IMGT | Kabat | AbM | Chothia | Contact |
|---|---|---|---|---|---|
| VH CDR1 | 27-38 | 31-35B | 26-35 | 26-32 | 30-35 |
| VH CDR2 | 56-65 | 50-65 | 50-58 | 53-55 | 47-58 |
| VH CDR3 | 105-117 | 95-102 | 95-102 | 96-101 | 93-101 |
| VL CDR1 | 27-38 | 24-34 | 24-34 | 26-32 | 30-36 |
| VL CDR2 | 56-65 | 50-56 | 50-56 | 50-52 | 46-55 |
| VL CDR3 | 105-117 | 89-97 | 89-97 | 91-96 | 89-96 |

The term “acceptor human framework” refers to a framework comprising the amino acid sequence of a light chain variable domain (V_{L}) framework, or a heavy chain variable domain (V_{H}) framework derived from a human immunoglobulin framework or a human consensus framework. An acceptor human framework “derived from” a human immunoglobulin framework or a human consensus framework may have the same amino acid sequence as the naturally occurring human immunoglobulin framework or human consensus framework, or it may have amino acid sequence changes compared to wild-type naturally occurring human immunoglobulin framework or human consensus framework. In some aspects, the number of amino acid changes are 10, 9, 8, 7, 6, 5, 4, 3, or 2, or 1. In some aspects, the V_{L} acceptor human framework is identical in sequence to the V_{L} human immunoglobulin framework sequence or human consensus framework sequence.

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

The term “epitope” or “antigenic determinant” refers to a site on an antigen (e.g., human FucGM1) to which an immunoglobulin or antibody specifically binds. Epitopes can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, or 22 amino acids in a unique spatial conformation.

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one aspect, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. Monoclonal antibodies include chimeric antibodies, human antibodies, and humanized antibodies and may occur naturally or be produced recombinantly.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one aspect, provided herein are single domain antibodies comprising two V_{H} domains with modifications such that single domain antibodies are formed.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain aspects, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A “human” antibody refers to an antibody having variable regions in which both the framework and CDR regions are derived from human germline immunoglobulin sequences. Furthermore, if the antibody contains a constant region, the constant region also is derived from human germline immunoglobulin sequences. Also encompassed are antibodies derived from human germline immunoglobulin sequences that include normal somatic hypermutations which alter the germline immunoglobulin sequences relative to the wild-type germline immunoglobulin sequences.

A “humanized” antibody refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Any additions, deletions, insertions, substitutions or modifications of amino acids are permissible as long as they do not abrogate the ability of the antibody to bind to a particular antigen. A “humanized” antibody may retain an antigenic specificity similar to that of the original antibody.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only.

A “chimeric antibody” refers to an antibody in which the variable regions are derived from one or more species and the constant regions are derived from another species, such as an antibody in which the variable regions are derived from a mouse antibody and the constant regions are derived from a human antibody. See U.S. Pat. No. 4,816,567; and Morrison et al., (1984) Proc. Natl. Acad. Sci. USA 81: 6851-6855.

A “domain antibody” or “nanobody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Bifunctional antibodies include, for example, heterodimeric antibody conjugates (e.g., two antibodies or antibody fragments joined together with each having different specificities), antibody/cell surface-binding molecule conjugates (e.g., an antibody conjugated to a non-antibody molecule such as a receptor), and hybrid antibodies (e.g., an antibody having binding sites for two different antigens).

A “multispecific antibody” is an antibody (e.g., bispecific antibodies, tri-specific antibodies) that recognizes two or more different antigens or epitopes.

The term “binds to the same epitope” is used with reference to two or more antibodies that bind to the same segment or same segments of amino acid residues. Techniques for determining whether antibodies bind to the same epitope may be determined by epitope mapping methods described herein. Other methods involve monitoring the binding of the antibody to antigen fragments (e.g., proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081), yeast display of mutant target sequence variants, or analysis of chimeras. In addition, computational combinatorial methods for epitope mapping can also be employed. These methods rely on the ability of the antibody of interest to affinity isolate specific short peptides from combinatorial phage display peptide libraries. Antibodies having the same V_{H} and V_{L} or the same CDR1, 2 and 3 sequences are expected to bind to the same epitope.

Antibodies that “compete with another antibody for binding to a target” refer to antibodies that inhibit (partially or completely) the binding of another antibody to the target. Whether two antibodies compete with each other for binding to a target, i.e., whether and to what extent one antibody inhibits the binding of the other antibody to a target, may be determined using known binding competition experiments involving surface plasmon resonance (SPR) and bio-layer interferometry (BLI). In certain aspects, an antibody competes with, and inhibits binding of another antibody to a target by at least 50%, 60%, 70%, 80%, 90% or 100%. The level of inhibition or competition may be different depending on which antibody is the “blocking antibody” (i.e., the antibody that when combined with an antigen blocks another immunologic reaction with the antigen). Competition assays can be conducted as described, for example, in Ed Harlow and David Lane, Cold Spring Harb. Protoc. 2006; doi:10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope, or to adjacent epitopes (e.g., as evidenced by steric hindrance). Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include competition for binding to cells expressing FucGM1, e.g., by flow cytometry. Other methods include surface plasmon resonance (SPR) (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁸ M, 10⁻⁹ M or 10⁻¹⁰ M or even lower when determined by, e.g., surface plasmon resonance (SPR) using a predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Any K_{D} greater than about 10⁴ M is generally considered to indicate nonspecific binding.

The term “kassoc” or “kₐ,” as used herein, refers to the association rate of a particular antibody-antigen interaction, whereas the term “k_{dis}” or “k_{d},” as used herein, refers to the dissociation rate of a particular antibody-antigen interaction. The term “K_{D},” as used herein, is intended to refer to the dissociation constant, which is obtained from the ratio of k_{d} to kₐ (i.e. k_{d}/kₐ) and is expressed as a molar concentration (M). K_{D} values for antibodies can be determined using methods well established in the art. A preferred method for determining the K_{D} of an antibody is by using surface plasmon resonance, for example, using a biosensor system such as a BIACORE® ® system or flow cytometry and Scatchard analysis, or bio-layer interferometry.

The term “EC50” or “IC50” in the context of an in vitro or in vivo assay using an antibody or immunoconjugate refers to the concentration of an antibody that induces a response that is 50% of the maximal response, i.e., halfway between the maximal response and the baseline. In pharmacology, the potency of a compound is expressed as the half-maximal effective concentration (EC50), which refers to the concentration of a drug that induces a response halfway between the baseline and maximum. While expressing the potency of a compound by its EC50 value makes sense in a clinical context, it is counterintuitive in the context of bioactivity-guided purification, as the potency of a compound is inversely related to its EC50 value, and the most potent compound is the one with the lowest EC50. Half-maximal inhibitory concentration (IC50) is the most widely used and informative measure of a drug's efficacy. It indicates how much drug is needed to inhibit a biological process by half, thus providing a measure of potency of an antagonist drug in pharmacological research.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, the term “conjugate” is used with reference to an immunoconjugate or antibody drug conjugate comprising an anti-FucGM1 antibody or antigen binding portion thereof described herein linked to a cytotoxic or therapeutic drug described herein.

The term “linker,” as used herein, refers to a chemical moiety comprising a covalent bond and/or any chain of atoms that may be used to covalently attach e.g., a drug to the antibody. Linkers are known in the art and include e.g., disulfide groups, thioether groups, acid labile groups, photolabile groups, peptidase labile groups and esterase labile groups. Conjugation of an antibody of the present disclosure with cytotoxic drugs or other growth inhibitory agents may be performed e.g. using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to an antibody (WO 94/11026).

In certain aspects, the linker is a “cleavable linker,” which may facilitate release of the cytotoxic drug or other growth inhibitory agent inside of or in the vicinity of a cell, e.g., a tumor cell. In some aspects, the linker is a linker cleavable in an endosome of a mammalian cell. For example, an acid-labile linker, a peptidase-sensitive linker, an esterase labile linker, a photolabile linker or a disulfide-containing linker (see e.g., U.S. Pat. No. 5,208,020) may be used.

The term “nucleic acid molecule,” as used herein, is used with reference to DNA molecules and RNA molecules. A nucleic acid molecule may be single-stranded or double-stranded, and may be a cDNA.

The term “isolated nucleic acid molecule,” as used herein in reference to nucleic acids encoding antibodies or antibody fragments (e.g., V_{H}, V_{L}, CDR3), is intended to refer to a nucleic acid molecule in which the nucleotide sequences are essentially free of other genomic nucleotide sequences, e.g., those encoding antibodies that bind antigens other than FucGM1, which other sequences may naturally flank the nucleic acid in human genomic DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

Also provided are “conservative sequence modifications” of the sequences set forth herein, e.g., amino acid sequence modifications which do not abrogate the binding of the antibody encoded by the nucleotide sequence or containing the amino acid sequence, to the antigen. Such conservative sequence modifications include conservative nucleotide and amino acid substitutions, as well as nucleotide and amino acid additions and deletions. For example, modifications can be introduced into a sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-FucGM1 antibody can be replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another aspect, mutations can be introduced randomly along all or part of an anti-FucGM1 antibody coding sequence, such as by saturation mutagenesis, and the resulting modified anti-FucGM1 antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, and at least about 98% to 99.5% of the amino acids.

The percent identity between two sequences is a function of the number of identical positions shared by the sequences (i.e., % homology=# of identical positions/total # of positions×100), considering the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The term “recombinant host cell” (or simply “host cell”), as used herein, is intended to refer to a cell that comprises a nucleic acid that is not naturally present in the cell and may be a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

The term “inhibition,” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity.

The term “immunotherapy” as used herein, refers to the treatment of a subject afflicted with, or at risk of contracting or suffering a recurrence of, a disease by a method comprising inducing, enhancing, suppressing or otherwise modifying an immune response.

The terms “immunostimulating therapy” and “immunostimulatory therapy,” as used herein, refer to a therapy that results in an increase (e.g., inducing or enhancing) an immune response in a subject for, e.g., treating cancer.

As used herein, “immune cell” refers to the subset of blood cells known as white blood cells, which include mononuclear cells such as lymphocytes, monocytes, macrophages, and granulocytes.

As used herein, “abnormal” is used in the context of the activity or level or expression of a molecule which is outside of the normal activity or expression level (e.g., overexpressed) as compared to e.g., a control sample or reference sample exhibiting a normal activity/expression profile. The term “normal” is used herein in the context of the activity or level of expression of a protein found in a population of healthy, gender- and age-matched subjects. The minimal size of this healthy population may be determined using standard statistical measures, e.g., the practitioner could consider the incidence of the disease in the general population and the level of statistical certainty desired in the results. In some aspects, the normal range for activity, level or expression of a biomarker is determined from a population of subjects (e.g., at least five, ten or twenty subjects), for example from a population of at least forty or eighty subjects, and from more than 100 subjects.

“T effector” (“T_{eff}”) cells refer to T cells (e.g., CD4+ and CD8+ T cells) with cytolytic activities as well as T helper (Th) cells, which secrete inflammatory cytokines and activate and direct other immune cells but does not include regulatory T cells (Treg cells).

As used herein, “administering” refers to the physical introduction of an ADC (comprising an anti-FucGM1 antibody or antigen binding portion thereof described herein linked to a cytotoxic moiety described herein via a linker alone or in combination with another therapeutic agent) to a subject, using any of the various methods and delivery systems known to those skilled in the art. Preferred routes of administration for ADCs described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intra-lymphatic, intralesional, intracapsular, intra-orbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, “cancer” refers to a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream, and includes a variety of cancers, including but not limited to e.g., carcinomas, melanomas, sarcomas, leukemias, lymphomas, germ cell tumors, and blastomas. Exemplary cancers for treatment include cancers of the brain, bladder, breast, cervix, colon, head and neck, kidney, lung, non-small cell lung, mesothelioma, ovary, prostate, stomach and uterus, leukemia, and medulloblastoma.

As used herein, the term “small molecule drug” refers to a molecular entity, often organic or organometallic, that is not a polymer, that has medicinal activity, and that has a molecular weight less than about 2 kilodaltons (kDa), less than about 1 kDa, less than about 900 daltons (Da), less than about 800 Da or less than about 700 Da. The term encompasses most medicinal compounds termed “drugs” other than protein or nucleic acids, although a small peptide or nucleic acid analog can be considered a small molecule drug. Examples include chemotherapeutic anticancer drugs and enzymatic inhibitors. Small molecule drugs can be derived synthetically, semi-synthetically (i.e., from naturally occurring precursors), or biologically.

The terms “treat,” “treating,” and “treatment,” as used herein, refer to any type of intervention or process performed on, or administering an active agent (e.g., ADC comprising an anti-FucGM1 antibody or antigen binding portion thereof described herein linked to a cytotoxic moiety via a linker as described herein) to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, “adjunctive” or “combined” administration (co-administration) includes simultaneous administration of an ADC (comprising an anti-FucGM1 antibody or antigen binding portion thereof described herein linked to a cytotoxic moiety described herein via a linker) and one or more additional agents and/or compounds in the same or different dosage form, or combined administration in separate dosages concurrently or sequentially. Thus, an ADC (comprising an anti-FucGM1 antibody or antigen binding portion thereof described herein linked to a cytotoxic moiety described herein via a linker), and second, third, or more agents and/or compounds (e.g., small molecules) can be simultaneously administered in a single formulation or formulated for separate administration and are administered concurrently or sequentially.

“Combination” therapy, as used herein, means administration of two or more therapeutic agents in a coordinated fashion, and includes, but is not limited to, concurrent and sequential dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. (See, e.g., Kohrt et al. (2011) Blood 117:2423). For example, the ADC (comprising an anti-FucGM1 antibody linked to a cytotoxic moiety described herein via a linker) can be administered first followed by (e.g., immediately followed by) the administration of a second agent (e.g., an antibody or antigen binding portion thereof), or vice versa. In one aspect, the ADC is administered prior to administration of the second agent (e.g., antibody or antigen binding portion thereof). In another aspect, the ADC is administered, for example, a few minutes (e.g., within about 30 minutes) or at least one hour of the second agent (e.g., antibody or antigen binding portion thereof). Such concurrent or sequential administration can result in both the ADC and the second agent being simultaneously present in treated patients.

The administration of effective amounts of the ADC (comprising an anti-FucGM1 antibody or antigen binding portion thereof described herein linked to a cytotoxic moiety described herein via a linker) alone, or the ADC combined with another compound or agent (e.g., an immune checkpoint inhibitor such as an anti-PD-1 antibody), according to any of the methods provided herein, can result in at least one therapeutic effect, including, for example, reduced tumor growth or size, reduced number of indicia of cancer (e.g., metastatic lesions) appearing over time, complete remission, partial remission, or stable disease. For example, the methods of treatment may produce a comparable clinical benefit rate (CBR=complete remission (CR)+partial remission (PR)+stable disease (SD) lasting ≥6 months) better than that achieved without administration of the ADC, or than that achieved with administration of any one of the ADC and the second agent, e.g., the improvement of clinical benefit rate is about 20% 20%, 30%, 40%, 50%, 60%, 70%, 80% or more.

As used herein, the terms “inhibit” and “block” (e.g., with regard to inhibition/blocking of FucGM1 binding or functional activity) are used interchangeably and encompass both partial and complete inhibition/blocking by the anti-FucGM1 antibody or fragment thereof in the ADC, or other inhibition/blocking of a functional activity by a therapeutic agent. The degree of inhibition may be at least 20%, 30%, 40%, 50%, 60%, 70%, 80%, 90%, 95%, or 100% (i.e., 2-fold or 2×), 3-fold, 5-fold or 10-fold relative to a control antibody or reference antibody. Additionally, the degree of inhibition may be between 20%-95%, 20%-80%, 20%-50%, 40%-95%, 40%-80%, 40%-60%, 50%-90%, 50%-70%, 75%-95%, 75%-85%, 2-fold to 20-fold, 2-fold to 10-fold, 2-fold to 5-fold, 4-fold to 12-fold, or 4-fold to 8-fold.

The term “effective dose” or “effective dosage” is defined as an amount sufficient to achieve or at least partially achieve a desired effect. A “therapeutically effective amount” or “therapeutically effective dosage” of a drug (e.g., ADC (comprising anti-FucGM1 antibody or antigen binding portion thereof described herein linked to a cytotoxic moiety described herein via a linker)) is any amount of the drug or therapeutic agent that, when used alone or in combination with another therapeutic agent, promotes disease regression evidenced by a decrease in severity of disease symptoms, an increase or therapeutic agent in frequency and duration of disease symptom-free periods, or a prevention of impairment or disability due to the disease affliction. A therapeutically effective amount or dosage of a drug or therapeutic agent includes a “prophylactically effective amount” or a “prophylactically effective dosage”, which is any amount of the drug or therapeutic agent that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, for the treatment of tumors, a therapeutically effective amount or dosage of the drug or therapeutic agent (e.g., ADC (comprising anti-FucGM1 antibody or antigen binding portion thereof described herein linked to a cytotoxic moiety described herein via a linker) inhibits tumor cell growth by at least about 20%, by at least about 30% by at least about 40%, by at least about 50%, by at least about 60%, by at least above 70%, by at least about 80%, or by at least about 90% relative to untreated subjects. In some aspects, a therapeutically effective amount or dosage of the drug or therapeutic agent completely inhibits cell growth or tumor growth, i.e., inhibits cell growth or tumor growth by 100%. The ability of a compound or therapeutic agent, including an antibody, to inhibit tumor growth can be evaluated using the assays described herein. Alternatively, this property of a composition comprising the compound or therapeutic agent can be evaluated by examining the ability of the composition to inhibit cell growth; such inhibition can be measured in vitro by assays known to the skilled practitioner.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, cats, dogs, cows, chickens, amphibians, and reptiles.

The term “sample” refers to tissue, bodily fluid, or a cell (or a fraction of any of the foregoing) taken from a patient or a subject. Normally, the tissue or cell will be removed from the patient, but in vivo diagnosis is also contemplated. In the case of a solid tumor, a tissue sample can be taken from a surgically removed tumor and prepared for testing. In the case of lymphomas and leukemias, lymphocytes, leukemic cells, or lymph tissues can be obtained (e.g., leukemic cells from blood) and appropriately prepared. Other samples, including e.g., urine, tears, serum, plasma, cerebrospinal fluid, feces, sputum, and cell extracts can also be useful for particular cancers.

The terms “detection” or “detected”, as used herein refer to qualitative and/or quantitative detection (measuring levels) with or without reference to a control.

The term “diagnosing”, as used herein, means the determination of the nature of a medical condition intended to identify a pathology which affects the subject from a number of collected data.

As used herein, “comprising” is synonymous with “including,” “containing,” “having” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to ±10% from the specified value. Unless otherwise indicated, all numbers expressing e.g., quantities of ingredients or properties (e.g., molecular weight, reaction conditions) described herein are to be understood as being modified by the term “about”.

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone.

As used herein, recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3% are expressly enumerated in this specification. These are only examples of what is specifically intended, and all possible combinations of numerical values between and including the lowest value and the highest value enumerated are to be considered to be expressly stated in this disclosure.

As used herein, the term “stereoisomer” refers to isomers of identical constitution that differ in the arrangement of their atoms in space. Enantiomers and diastereomers are examples of stereoisomers. Geometric isomers are also examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity. Geometric isomers of C═C double bonds can also be present in the ADCs, and all such stable isomers are contemplated in the present invention. Cis- and trans- (or E- and Z-) geometric isomers of the ADCs of the present invention are described and may be isolated as a mixture of isomers or as separated isomeric forms.

Various aspects described herein are described in further detail in the following subsections.

### II. Anti-Fucosyl-GM1 Antibodies

Human monoclonal antibodies (HuMAbs) that bind specifically to fucosyl-GM1 with high affinity have been disclosed in U.S. Pat. No. 8,383,118 and WO 2007/067992 (e.g., human monoclonal antibodies 5B1, 5B1a, 7D4, 7E4, 13B8 and 18D5). Each of the HuMAbs disclosed in U.S. Pat. No. 8,383,118 has been demonstrated to exhibit one or more desirable functional properties: (1) specifically binds to fucosyl-GM1; (2) binds to fucosyl-GM1 with high affinity (for example with a K_{D} of 1×10⁻⁷ M or less); (c) binds to the human small cell lung cancer cell line DMS-79 (Human SCLC ATCC #CRL-2049); and (d) inhibit growth of tumor cells in vitro or in vivo. Preferably, the antibody binds to fucosyl-GM1 with a K_{D} of 5×10⁻⁸ M or less, binds to fucosyl-GM1 with a K_{D} of 1×10⁻⁸ M or less, binds to fucosyl-GM1 with a K_{D} of 5×10⁻⁹ M or less, or binds to fucosyl-GM1 with a K_{D} of between 1×10⁻⁸ M and 1×10⁻¹⁰ M or less. Standard assays to evaluate the binding ability of the antibodies toward fucosyl-GM1 are known in the art, including for example, ELISAs, Western blots and RIAs. The binding kinetics (e.g., binding affinity) of the antibodies also can be assessed by standard assays known in the art, such as by ELISA, Scatchard and Biacore analysis.

In some aspects, an anti-fucosyl-GM1 Ab comprises BMS-986012 (also referred to as MBN-001; MDX-1110 or 7E4).

Anti-fucosyl-GM1 Abs usable in the disclosed methods also include isolated Abs that bind specifically to fucosyl-GM1 and cross-compete for binding to fucosyl-GM1 with BMS-986012 (see, e.g., U.S. Pat. No. 8,383,118; WO 2007/067992). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of BMS-986012 by virtue of their binding to the same epitope region of fucosyl-GM1. Cross-competing Abs can be readily identified based on their ability to cross-compete with BMS-986012 in standard fucosyl-GM1 binding assays such as Biacore analysis, ELISA assays or flow cytometry (see, e.g., WO 2013/173223).

For administration to human subjects, these antibodies are preferably chimeric antibodies, or more preferably humanized or human antibodies. Such chimeric, humanized or human mAbs can be prepared and isolated by methods well known in the art. Anti-fucosyl-GM1 antibodies usable in the methods of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody. Anti-fucosyl-GM1 antibodies (or V_{H} and/or V_{L} domains derived therefrom) suitable for use in the invention can be generated using methods well known in the art.

An exemplary anti-fucosyl-GM1 antibody is MBN-001 comprising heavy and light chains comprising the sequences shown in SEQ ID NOs: 3 and 4, respectively, or antigen binding fragments and variants thereof.

In other aspects, the antibody has heavy and light chain CDRs or variable regions of MBN-001. Accordingly, in one aspect, the antibody comprises CDR1, CDR2, and CDR3 domains of the VH of MBN-001 having the sequence set forth in SEQ ID NO: 1, and CDR1, CDR2 and CDR3 domains of the VL of MBN-001having the sequence set forth in SEQ ID NO: 2. In some aspects, the antibody comprises CDR1, CDR2, and CDR3 domains of the VH of MBN-001 having the sequence set forth in SEQ ID NO: 1 and CDR1, CDR2 and CDR3 domains of the VL of MBN-001 having the sequence set forth in SEQ ID NO: 2.

In another aspect, the antibody comprises heavy chain CDR1, CDR2 and CDR3 domains comprising the sequences set forth in SEQ ID NOs: 5, 6, and 7, respectively.

In some aspects, the antibody comprises the light chain CDR1, CDR2 and CDR3 domains comprising the sequences set forth in SEQ ID NOs: 8, 9, and 10, respectively.

In some aspect, the antibody comprises heavy chain CDR1, CDR2 and CDR3 domains comprising the sequences set forth in SEQ ID NOs: 5, 6, and 7, respectively and light chain CDR1, CDR2 and CDR3 domains comprising the sequences set forth in SEQ ID NOs: 8, 9, and 10, respectively.

In another aspect, the antibody comprises a VH region comprising the amino acid sequences set forth in SEQ ID NO: 1. In some aspects, the antibody comprises a VL region comprising the amino acid sequence set forth in SEQ ID NO: 2. In some aspects, the antibody comprises a VH region comprising the amino acid sequences set forth in SEQ ID NO: 1 and a VL region comprising the amino acid sequences set forth in SEQ ID NO: 2.

In another aspect, the antibody competes for binding with and/or binds to the same epitope on fucosyl-GM1 as the above-mentioned antibodies.

In another aspect, the antibody has at least about 80% variable region amino acid sequence identity with the above-mentioned antibodies (e.g., at least about 80%, at least about 85%, at least about 90%, at least about 95%, at least about 96%, at least about 97%, at least about 98%, or 99% variable region identity with SEQ ID NO: 1 or SEQ ID NO: 2). In some aspects, the antibody comprises a heavy chain comprising the sequence set forth in SEQ ID NO: 3. In some aspects, the antibody comprises a light chain comprising the sequence set forth in SEQ ID NO: 4. In some aspects, the antibody comprises a heavy chain comprising the sequence set forth in SEQ ID NO: 3 and a light chain comprising the sequence set forth in SEQ ID NO: 4.

### Antibody Engineering of the Fc Region

The ADC or its components (e.g., anti-FucGM1 antibody or antigen binding portion thereof) described herein may include modifications to their respective Fc regions, typically to alter one or more of their physical or functional properties, such as effector function (e.g., antigen-dependent cellular cytotoxicity), Fc receptor binding, serum half-life, and complement fixation). Furthermore, the ADC or its components (e.g., anti-FucGM1 antibody or antigen binding portion thereof) can be chemically modified (e.g., one or more chemical moieties can be attached to the antibody) or be modified to alter its glycosylation, again to alter one or more properties of the antibody or fragment. In the context of Fc region modifications, the numbering of residues in the Fc region is that of the EU index of Kabat.

The ADC or its components (e.g., anti-FucGM1 antibody or antigen binding portion thereof) disclosed herein also include antibodies and fragments with modified (or blocked) Fc regions to provide altered effector functions as described in e.g., U.S. Pat. No. 5,624,821; U.S. Patent Publication numbers US2009/280114 and US2011/142858; and PCT Publication Number WO2006/0057702. Such modifications can further include alterations to enhance or suppress various reactions of the immune system, with possible beneficial effects in diagnosis and therapy.

### Altered Effector Functions

In some aspects, the ADC or its components (e.g., anti-FucGM1 antibody or antigen binding portion thereof) comprises a variant Fc region that is modified (e.g., by amino acid substitution, deletion and/or insertion) relative to a parent Fc sequence (e.g., an unmodified Fc polypeptide that is subsequently modified to generate a variant) to increase or reduce the ability of the antibody or antigen-binding portions thereof to mediate one or more effector function(s) and/or to increase or decrease its binding to the Fc-gamma receptors (FcγRs), while retaining its antigen binding ability. Thus, in some aspects, the ADC or its components (e.g., anti-FucGM1 antibody or antigen binding portion thereof) may include one or more amino acid changes altering affinity for an effector ligand, such as an Fc receptor or the C1 component of complement. This approach is described in further detail in U.S. Pat. Nos. 5,624,821 and 5,648,260.

The interaction between the constant region of an antigen binding protein (such as an anti-FucGM1 antibody, or antigen binding portion thereof,) and various Fc receptors (FcR), including FcγRI (CD64), FcγRII (CD32) and FcγRIII (CD16), is believed to mediate the effector functions, such as ADCC and CDC, of the antigen binding protein. The Fc receptor is also important for antibody cross-linking, which can be important for anti-tumor immunity. In some aspects, modifications can be made in the Fc region in order to generate an Fc variant promoting (a) increased or decreased antibody-dependent cell-mediated cytotoxicity (ADCC), (b) increased or decreased complement mediated cytotoxicity (CDC), (c) increased or decreased affinity for C1q, (d) increased or decreased affinity for a Fc receptor relative to the parent Fc, and/or (e) increased or decreased pharmacokinetic stability.

Alterations of the Fc region may include amino acid changes, such as substitutions, deletions, insertions, glycosylation, deglycosylation, and/or addition of multiple Fc regions. Combining amino acid modifications may be particularly desirable. For example, the variant Fc region may include two, three, four, five, or more substitutions therein, e.g., of the specific Fc region positions identified herein. In some aspects, the Fc region is altered by replacing at least one amino acid residue with a different amino acid residue such that the antibody has an altered affinity for an effector ligand but retains the antigen-binding ability of the parent antibody. For example, one or more amino acids selected from amino acid residues 234, 235, 236, 237, 297, 318, 320 and 322 can be replaced with a different amino acid residue. The effector ligand to which affinity is altered can be, for example, an Fc receptor or the C1 component of complement. This approach is described in further detail in U.S. Pat. Nos. 5,624,821 and 5,648,260, both by Winter et al. In some aspects, the C1q binding site may be removed from the Fc region by deleting or substituting, for example, the EKK sequence of human IgG1. In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al. In another example, one or more amino acid residues within amino acid positions 231 and 239 are altered to change the ability of the antibody to fix complement. This approach is described further in U.S. Pat. No. 6,180,377. In some aspects, provided herein are effector function-less versions of the anti-FucGM1 antibodies or antigen binding portions thereof having e.g., a mutant hIgG1f allotype (hIgG1.3f) comprising the amino acid sequence set forth in SEQ ID NO: 171. The hIgG1.3f variant is a triple mutant version of hIgG1f (L234A, L235E, G237A) which lacks FcγR binding and effector function.

In some aspects, the ADC or its components (e.g., anti-FucGM1 antibody or antigen binding portion thereof) may be engineered to have different affinities and selectivities for Fc gamma receptors (FcγRs) by mutating the heavy chain constant region, including the hinge and Fc domains. Mutations can be introduced to either enhance or reduce FcγR binding. These mutations can increase or decrease FcγR-mediated cross-linking and/or signaling. For therapeutic targets, such as FucGM1, FcγR-mediated cross-linking of anti-FucGM1 antibodies have the potential to provide undesirable agonist signaling and potential for toxicity absent the introduction of certain modification to obviate this problem.

Binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In some aspects, the ADC or its components (e.g., anti-FucGM1 antibody or antigen binding portion thereof) may be engineered for reduced FcγR binding and potential for cross-linking and/or signaling, specifically, reduced engagement of the “low affinity” FcγRs hCD32a/FcγRIIa, hCD32b/FcγRIIb, hCD16a/FcγRIIIa, and hCD16b/FcγRIIIb. Engagement of the “high affinity” receptor CD64/FcγRI is generally believed to be of lower concern due to saturation of this receptor with serum IgG. Therefore, in some aspects, the ADC or its components (e.g., anti-FucGM1 antibody or antigen binding portion thereof) may comprise an IgG1.3 Fc region, which is essentially devoid of binding to CD16, CD32a, CD32b and CD64 and lacks ADCC, ADCP and CDC functions (see U.S. Pat. No. 10,077,306 and U.S. Patent Publication No. US2022/0106400). In some aspects, the Fc region may be engineered for increased ADCC and/or increased FcγR binding by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (as described e.g., in U.S. Pat. No. 6,737,056) wherein the numbering of the residues in the Fc region is that of the EU index as in Kabat. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 247I, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

In some aspects, the Fc region is modified to decrease the ability of the anti-FucGM1 antibody or antigen binding portion thereof described herein to mediate effector function and/or to increase anti-inflammatory properties by modifying residues 243 and 264. In one aspect, the Fc region of the ADC or its components (e.g., anti-FucGM1 antibody or antigen binding portion thereof) is modified by changing the residues at positions 243 and 264 to alanine. In another aspect, the Fc region is modified to decrease the ability of the ADC or its components (e.g., anti-FucGM1 antibody or antigen binding portion thereof) to mediate effector function and/or to increase anti-inflammatory properties by modifying residues 243, 264, 267 and 328.

Other Fc modifications to the Fc region include those for reducing or ablating binding to FcγRs and/or complement proteins, thereby reducing or ablating Fc-mediated effector functions, such as ADCC, ADCP, and CDC. Modifications for altering binding to FcγRllb include one or more substitutions, insertions, and deletions at positions 234, 235, 236, 237, 239, 266, 267, 268, 269, 325, 326, 327, 328, and 332, wherein numbering is according to the EU index. In one aspect, the Fc variants provide selectively enhanced affinity to FcγRllb relative to one or more activating receptors. Exemplary substitutions include but are not limited to 234G, 235G, 236R, 237K, 267R, 269R, 325L, and 328R. Other Fc variants for enhancing binding to FcγRllb include 235Y/267E, 236D/267E, 236R/328R, 239D/268D, 239D/267E, 267E/268D, 267E/268E, and 267E/328F. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

In certain aspects, the Fc region may be modified to remove an ADCC site.

ADCC sites can be found, for example, in Molec. Immunol. 29 (5): 633-9 (1992) with regard to ADCC sites in IgG1. In addition, IgG1 mutants containing L235V, F243L, R292P, Y300L and P396L mutations were found to exhibit enhanced binding to FcγRIIIa and concomitantly enhanced ADCC activity in transgenic mice expressing human FcγRIIIa in models of B cell malignancies and breast cancer (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S. Specific examples of variant Fc domains are disclosed for example, in U.S. Pat. No. 6,096,871 and PCT Publication number WO 97/34631.

Optionally, the Fc region may comprise a non-naturally occurring amino acid residue at additional and/or alternative positions (see, e.g., U.S. Pat. Nos. 5,624,821; 6,277,375; 6,737,056; 6,194,551; 7,317,091; 8,101,720; PCT Patent Publication numbers WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In one aspect, the hinge region of Fc is modified such that the number of cysteine residues in the hinge region is altered, e.g., increased or decreased. For example, in one aspect, the number of cysteine residues in the hinge region of CH1 is increased to provide increased the stability of the antibody or decreased to provide enhanced assembly of the light and heavy chains or as described in U.S. Pat. No. 5,677,425.

In some aspects, the changes to the Fc region may be made to increase the biological half-life of the ADC so as to facilitate less frequent dosing, with the concomitant increase convenience and decreases use of material (Presta (2005) J. Allergy Clin. Immunol. 116:731 at 734-35). Various approaches may be employed. For example, in certain aspects, this may be achieved by increasing the binding affinity of the Fc region for the neonatal Fc receptor (FcRn). For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

Other Fc variants for increased binding to FcRn and/or improved pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (Dall'Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

In another aspect, the Fc hinge region may be mutated to decrease the biological half-life of the antibody or fragment. For example, one or more amino acid mutations may be introduced into the CH2-CH3 domain interface region of the Fc hinge fragment such that the antibody or fragment has impaired Staphylococcal protein A (SpA) binding relative to native Fc-hinge domain SpA binding as described in U.S. Pat. No. 6,165,745.

In certain aspects, hybrid IgG isotypes with particular biological characteristics may be used. For example, in certain aspects, one or more regions and/or mutations from an IgG2 or IgG4. In one aspect, the ADC described herein comprises an IgG4 isotype antibody or fragment comprising a serine to proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system). When using an IgG4 constant domain, it is usually to include the substitution S228P, which mimics the hinge sequence in IgG1 and thereby stabilizes IgG4 molecules.

In another aspect, an IgG1/IgG3 hybrid variant may be constructed by substituting IgG1 positions in the CH2 and/or CH3 region with the amino acids from IgG3 at positions where the two isotypes differ. Thus, a hybrid variant IgG antibody may be constructed that comprises one or more substitutions, e.g., 274Q, 276K, 300F, 339T, 356E, 358M, 384S, 392N, 397M, 4221, 435R, and 436F. In other aspects described herein, an IgG1/IgG2 hybrid variant may be constructed by substituting IgG2 positions in the CH2 and/or CH3 region with amino acids from IgG1 at positions where the two isotypes differ. Thus, a hybrid variant IgG antibody may be constructed that comprises one or more substitutions, e.g., one or more of the following amino acid substitutions: 233E, 234L, 235L, 236G (referring to an insertion of a glycine at position 236), and 327A.

In some aspects, the variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other aspects, the Fc region may be modified to make it more compatible with a selected host cell. For example, one may remove the PA sequence near the N-terminus of a typical native Fc region, which may be recognized by a digestive enzyme in E. coli such as proline iminopeptidase.

The ADC or its components (e.g., anti-FucGM1 antibody or antigen binding portion thereof) disclosed herein may contain one or more glycosylation sites. Such glycosylation sites may result in increased immunogenicity of the antibody or fragment or an alteration of the pK of the antibody due to altered antigen-binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N—X—S/T sequence. Therefore, in some aspects, the glycosylation properties of the ADC or its components (e.g., anti-FucGM1 antibody or antigen binding portion thereof) described herein may be modified. For example, one or more glycosylation sites within the Fc domain may be modified or removed. Residues that are typically glycosylated (e.g., asparagine) may confer a cytolytic response. Such residues may be deleted or substituted with unglycosylated residues (e.g., alanine) to produce an aglycosylated antibody. In certain aspects, glycosylation can be altered to, for example, increase the affinity of the antibody for antigen. For example, one or more amino acid substitutions can be made that result in elimination of one or more variable region framework glycosylation sites to thereby eliminate glycosylation at that site. The resulting aglycosylation may increase the affinity of the antibody for antigen. Such an approach is described in further detail in U.S. Pat. Nos. 5,714,350 and 6,350,861 by Co et al. Glycosylation of the constant region on N297 may be prevented by mutating the N297 residue to another residue, e.g., N297A, and/or by mutating an adjacent amino acid, e.g., 298 to thereby reduce glycosylation on N297. Additionally, or alternatively, an ADC or its components (e.g., anti-FucGM1 antibody or antigen binding portion thereof) described herein can be engineered with an altered type of glycosylation, such as a hypofucosylated antibody having reduced amounts of fucosyl residues or an antibody having increased bisecting GlcNac structures. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. Such carbohydrate modifications can be accomplished by, for example, expressing the antibody in a host cell with altered glycosylation machinery.

Cells with altered glycosylation machinery can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase (i.e., alpha-1,6-fucosyltransferase), such that antibodies expressed in such a cell line exhibit hypofucosylation. Recombinant host cells which have been genetically modified to inactivate the FUT8 gene encoding an alpha-1,6-fucosyltransferase are available. See, e.g., the POTELLIGENT™ technology system available from BioWa, Inc. (Princeton, N.J.) in which CHOK1SV cells lacking a functional copy of the FUT8 gene produce monoclonal antibodies having enhanced ADCC activity that is increased relative to an identical monoclonal antibody produced in a cell with a functional FUT8 gene. Aspects of the POTELLIGENT™ technology system are described in U.S. Pat. Nos. 7,214,775 and 6,946,292, and PCT Publication numbers WO00/61739 and WO02/31240.

PCT Publication number WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication number WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies described herein is pegylation. In some aspects, the ADC or its components (e.g., anti-FucGM1 antibody or antigen binding portion thereof) described herein is pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some aspects, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain aspects, the antibody to be pegylated is an aglycosylated antibody. Methods for pegylating proteins can be applied to the antibodies or antigen binding portions thereof described herein. See for example, European patent number EP 0 154 316 by Nishimura et al. and European patent number EP 0 401 384 by Ishikawa et al.

Effector functions can be measured in a number of ways including for example via binding of the FcγRIII to Natural Killer cells or via FcγRI to monocytes/macrophages to measure for ADCC effector function. For example, an antigen binding protein of the present invention can be assessed for ADCC effector function in a Natural Killer cell assay. Examples of such assays can be found in Shields et al., 2001 J. Biol. Chem., Vol. 276, p 6591-6604; Chappel et al., 1993 J. Biol. Chem., Vol 268, p 25124-25131; Lazar et al., 2006 PNAS, 103; 4005-4010.

The affinities and binding properties of an Fc region for its ligand may be determined by a variety of in vitro assay methods (biochemical or immunological based assays) including, but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

With regard to the above-described modifications for increasing or decreasing one or more of the functional properties described herein (e.g., biochemical, immunochemical, cellular, physiological or other biological activities, as determined using methods known to the art and described herein), the resulting increase in a given parameter may represent a statistically significant increase of at least 10% of the measured parameter, for example at least 20%, 30%, 40%, 50%, 60%, 70%, 80%, 90%, 95%, or 100% (i.e., 2-fold), 3-fold, 5-fold or 10-fold. Conversely, the resulting decrease in a measured parameter may represent a statistically significant decrease of at least 10% of the measured parameter, e.g., at least 20%, 30%, 40%, 50%, 60%, 70%, 80%, 90%, 95%, or 100%, 3-fold, 5-fold or 10-fold.

Any of the above-described modifications may be employed alone or in combination with any of the above-described modifications or those described in the next section in order to further enhance or decrease effector functions or other desirable properties (e.g., stability, expression).

### Antibody Engineering of Variable Regions

In some aspects, the ADC or its components (e.g., anti-FucGM1 antibody or antigen binding portion thereof) are engineered with modifications to framework residues within the variable domains of the parental antibody, e.g., to improve the properties of the antibody or antigen binding portion thereof. Typically, such framework modifications are made to decrease the immunogenicity of the anti-FucGM1 antibodies or antigen binding portions thereof. This is usually accomplished by replacing non-CDR residues in the variable domains (i.e., framework residues) in a parental (e.g., rodent) antibody with analogous residues from the immune repertoire of the species in which the antibody is to be used, e.g., human residues in the case of human therapeutics. Such an antibody is referred to as a “humanized” antibody. In some cases, it is desirable to increase the affinity, or alter the specificity of an engineered (e.g., humanized) antibody. One approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another approach is to revert to the original parental (e.g., rodent) residue at one or more positions of the engineered (e.g., humanized) antibody, e.g., to restore binding affinity that may have been lost in the process of replacing the framework residues. (See, e.g., U.S. Pat. Nos. 5,693,762, 5,585,089 and 5,530,101.) In certain aspects, the ADCs comprising the anti-FucGM1 antibodies and antigen binding portions thereof in an ADC are engineered (e.g., humanized) to include modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al., (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR”s or “hyper variable” loops.

Commercial scientific software such as MOE (Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent-exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689. In certain aspects, one or more glycosylation sites in either the light or heavy chain immunoglobulin variable regions, such as the framework regions, may be modified or removed to reduce immunogenicity. In particular aspects, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain aspects, the antibodies provided herein do not contain deamidation or asparagine isomerism sites. For example, an asparagine (Asn) residue may be changed to Gln or Ala to reduce the potential for formation of isoaspartate at any Asn-Gly sequences, particularly within a CDR.

A similar problem may occur at an Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734.

In various aspects, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen-binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for FucGM1, or other desired biological activity to unacceptable levels. See Table 2 for exemplary stabilizing CDR variants.

TABLE 2
Exemplary stabilizing CDR variants

| CDR Residue | Stabilizing Variant Sequence |
|---|---|
| Asn-Gly (N-G) | Gln-Gly, Ala-Gly, or Asn-Ala (Q-G), (A-G), |
| | or (N-A) |
| Asp-Gly | Glu-Gly, Ala-Gly or Asp-Ala |
| (D-G) | (E-G), (A-G), or (D-A) |
| Met | Lys, Leu, Ala, or Phe |
| (M) | (K), (L), (A), or (F) |
| Asn (N) | Gln or Ala |
| | (Q) or (A) |
| Asn-Pro (N-P) | Gln-Pro, Ala-Pro, or Asn-Ala (Q-P), |
| | (A-P), or (N-A) |

### III. Antibody Drug Conjugates

The present disclosure provides an antibody drug conjugate (ADC) comprising an anti-FucGM1 antibody or antigen binding portion thereof described herein which is linked or conjugated via a phosphorus (V) moiety (also denoted as “P5”) and a linker to a cytotoxic moiety, i.e., topoisomerase inhibitors such as camptothecin or derivatives and analogs thereof.

Some aspects of the disclosure are directed to an ADC comprising an anti-fucosyl-GM1 antibody, or an antigen-binding portion thereof, and a topoisomerase I inhibitor, wherein the anti-fucosyl-GM1 antibody, or an antigen-binding portion thereof, comprises a heavy chain variable region (VH) and a light chain variable region (VL), wherein the VH comprises complementarity determining region (CDR) 1 (CDRH1) comprising the amino acid sequence as set forth in SEQ ID NO: 5, CDRH2 comprising the amino acid sequence as set forth in SEQ ID NO: 6, and CDRH3 comprising the amino acid sequence as set forth in SEQ ID NO: 7 and the VL comprises CDRL1 comprising the amino acid sequence as set forth in SEQ ID NO: 8, CDRL2 comprising the amino acid sequence as set forth in SEQ ID NO: 9, and CDRL3 comprising the amino acid sequence as set forth in SEQ ID NO: 10, and wherein the topoisomerase I inhibitor comprises exatecan, deruxtecan, or derivatives and analogs thereof.

Some aspects of the disclosure are directed to an ADC comprising: a) an anti-fucosyl-GM1 antibody, or an antigen-binding portion thereof, and b) a topoisomerase I inhibitor.

In some aspects, the topoisomerase I inhibitor comprises deruxtecan, or derivatives and analogs thereof. In some aspects, the ADC has the formula (I) shown below, wherein Ab is the anti-fucosyl-GM1 antibody, or an antigen-binding portion thereof.

Certain aspects of the disclosure are directed to an antibody drug conjugate (ADC) comprising an anti-fucosyl-GM1 antibody, or an antigen-binding portion thereof, and a topoisomerase I inhibitor, wherein the ADC comprises formula (I) shown below, or a pharmaceutically acceptable salt, a stereoisomer, or a solvate thereof.

In some aspects, the ADC of the present disclosure has the formula (I):

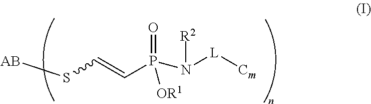

or a pharmaceutically acceptable salt, a stereoisomer, or a solvate thereof, wherein:
AB is an anti-FucGM1 antibody or an antigen-binding portion thereof discloses herein;
[symbol] indicates that the configuration of the double bond may be E or Z;
R¹ is a polyalkylene glycol unit comprising at least 3 alkylene glycol subunits;
R² is H or an optionally substituted aliphatic or aromatic residue;
L is a linker;
C is a cytotoxic moiety;
m is an integer ranging from 1 to 10; and
n ranges from 1 to 20.

In some aspects, AB comprises the anti-fucosyl-GM1 antibody, or an antigen-binding portion thereof, which comprises a heavy chain variable region (VH) and a light chain variable region (VL), wherein the VH comprises complementarity determining region (CDR) 1 (CDRH1) comprising the amino acid sequence as set forth in SEQ ID NO: 5, CDRH2 comprising the amino acid sequence as set forth in SEQ ID NO: 6, and CDRH3 comprising the amino acid sequence as set forth in SEQ ID NO: 7 and the VL comprises CDRL1 comprising the amino acid sequence as set forth in SEQ ID NO: 8, CDRL2 comprising the amino acid sequence as set forth in SEQ ID NO: 9, and CDRL3 comprising the amino acid sequence as set forth in SEQ ID NO: 10.

In some aspects, C is a cytotoxic moiety. The term “cytotoxic moiety” or sometimes “payload,” refer to a chemical or biochemical moiety that is conjugated to the anti-FucGM1 antibody via a linker. Preferably, the cytotoxic moiety is a camptothecin moiety. More preferably, the cytotoxic moiety is a camptothecin derivative, exatecan.

In some aspects, the cytotoxic moiety is topoisomerase I inhibitor produced by nature, camptothecin or a derivative or analog thereof. In some aspects, the cytotoxic moiety is a camptothecin derivative, e.g., exatecan. The structure of exatecan is shown below.

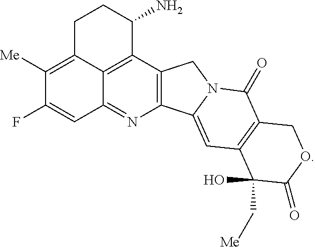 [SMILES: CC[C@@]1(O)C(=O)OCc2c1cc1n(c2=O)Cc2c-1nc1cc(F)c(C)c3c1c2[C@@H](N)CC3]

All stereoisomers of the exatecan are contemplated for the ADCs disclosed herein.

In some aspects, the linker and drug moiety (e.g., payload, e.g., cytotoxic moiety) is a structure/compound shown below:

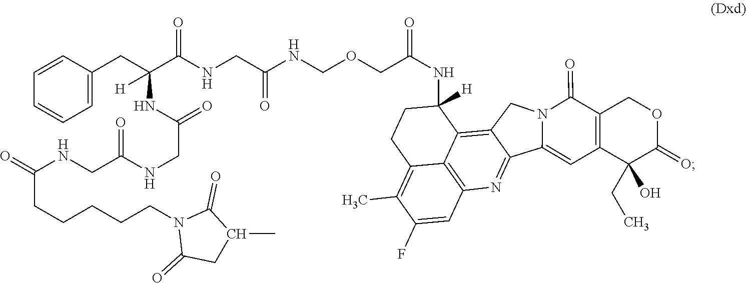 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

In some aspects, the ADC of the present disclosure has the formula (I):

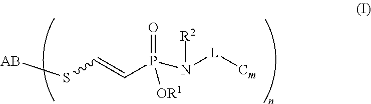

or a pharmaceutically acceptable salt, a stereoisomer, or a solvate thereof, wherein:
AB is an anti-FucGM1 antibody or an antigen-binding portion thereof discloses herein;
[symbol] indicates that the configuration of the double bond may be E or Z;
R¹ is a polyalkylene glycol unit comprising at least 3 alkylene glycol subunits;
R² is H or an optionally substituted aliphatic or aromatic residue;
L is a linker;
C is exatecan;
m is an integer ranging from 1 to 10;
n ranges from 1 to 20; and
AB comprises the anti-fucosyl-GM1 antibody, or an antigen-binding portion thereof, which comprises a VH and a VL, wherein the VH comprises CDR 1 (CDRH1) comprising the amino acid sequence as set forth in SEQ ID NO: 5, CDRH2 comprising the amino acid sequence as set forth in SEQ ID NO: 6, and CDRH3 comprising the amino acid sequence as set forth in SEQ ID NO: 7 and the VL comprises CDRL1 comprising the amino acid sequence as set forth in SEQ ID NO: 8, CDRL2 comprising the amino acid sequence as set forth in SEQ ID NO: 9, and CDRL3 comprising the amino acid sequence as set forth in SEQ ID NO: 10.

In some aspects, R¹ comprises 3 to 100 subunits having the structure:

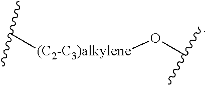

In some aspects, R¹ is

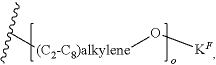

wherein: [symbol] indicates the position of the O; K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; and o is an integer ranging from 3 to 100.

In some aspects, R¹ comprises 3 to 50 subunits having the structure:

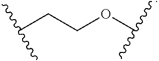 [SMILES: "." ; *CCO*]

In some aspects, R¹ is

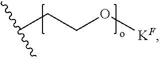

wherein [symbol] indicates the position of the O; K^{F} is selected from the group consisting of —H, —(C₁-C₁₀)alkyl, and —(C₂-C₁₀)alkyl-OH; and o is an integer ranging from 3 to 50.

In some aspects, K^{F} is H.

In some aspects, o is an integer ranging from 8 to 30, e.g., from 8 to 16 or from 20 to 28, e.g., 10, 11, 12, 13 14, 22, 23, 24, 25, or 26.

In some aspects, the linker L is cleavable.

In some aspects, the linker L is cleavable by a protease, a glucuronidase, a sulfatase, a phosphatase, an esterase, or by disulfide reduction.

In some aspects, the linker is cleaved under physiological conditions, in particular inside a cell by e.g., a lysosomal or endosomal protease to release the attached cytotoxic moiety. In some aspects, the cleavable linkers are designed to release the free cytotoxic moiety in an unmodified form. Cleavable linkers include, e.g., disulfide linkers, acid labile linkers, photolabile linkers, peptidase labile linkers, and esterase labile linkers. Typically, a peptidyl linker is at least two amino acids long or at least three amino acids long.

Peptidase labile linkers can be used to cleave certain peptides inside or outside cells. In one aspect, the cleavable linker is cleaved under mild conditions, i.e., conditions within a cell under which the activity of the cytotoxic moiety is not affected.

Depending on the linker design, membrane permeable (lipophilic) toxins that are released inside target positive cells can pass the cell membrane and kill other cells that are in close proximity, including neighboring cancer cells that lack antigen expression (bystander effect) (Kovtun, Y. V. et al. (2006) Cancer Res. 66 (6), 3214-3221). The ability of such cytotoxic drugs to mediate local bystander killing is one selection criterium for the ADCs according to the present disclosure.

Cleaving agents can include e.g., cathepsins B and D and plasmin, all of which are known to hydrolyze dipeptide drug derivatives resulting in the release of active drug inside target cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). In specific aspects, the peptidyl linker cleavable by an intracellular protease is a valine-citrulline (Val-Cit) linker or a phenylalanine-lysine (Phe-Lys) linker. One advantage of using intracellular proteolytic release of the therapeutic agent is that the agent is typically attenuated when conjugated and the serum stabilities of the conjugates are typically high.

A variety of linkers may be used in the conjugates described herein. In some aspects, the linker comprises a peptidyl linker, such as dipeptide valine (Val)-citrulline (Cit) (vc), which can be cleaved by cathepsin inside tumor cells. Additional peptidyl linkers include, but are not limited to Val-Cit, Ala-Val, Val-Ala-Val, Lys-Lys, Pro-Val-Gly-Val-Val (SEQ ID NO: 100), Ala-Asn-Val, Val-Leu-Lys, Ala-Ala-Asn, Cit-Cit, Val-Lys, Lys, Cit, Ser, or Glu. In some aspects, the linker L is cleavable by a protease, for example cathepsins B and D.

In some aspects, the linker L has the formula:

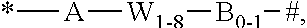

wherein unit A is a first spacer unit; W is an amino acid; B is a second spacer unit, * denotes the attachment point to the —Y— and # denotes the attachment point to the cytotoxic moiety. In some aspects, the cytotoxic moiety is exatecan.

In some aspects, the first spacer unit A has the structure:

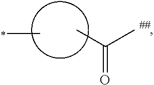

wherein

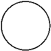

is a 5- or 6-membered carbocycle; * denotes the attachment point to —Y— and ## denotes the attachment point to W. A preferred unit A is

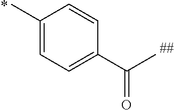

In some aspects, W is a dipeptide (W₂).

In some aspects, the dipeptide is selected from the group consisting of valine-citrulline (Val-Cit) and valine-alanine (Val-Ala).

In some aspects, the dipeptide is Val-Cit.

In some aspects, B is a PAB group having the following structure:

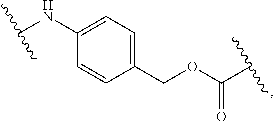 [SMILES: *Nc1ccc(COC(*)=O)cc1 ; ","]

wherein the NH group is bonded to —W— and the C(O) group is bonded to the cytotoxic moiety.

In some aspects, the linker has the following structure:

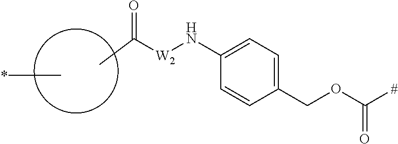

wherein W₂ is a dipeptide,
* indicates the attachment point to the Y, and # indicates the attachment point to the cytotoxic moiety.

In some aspects, the linker has the following structure:

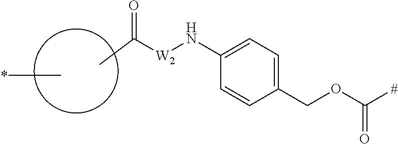

wherein

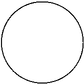

is a 5- or 6-membered carbocycle, W₂ is a dipeptide, * indicates the attachment point to the Y, and # indicates the attachment point to the cytotoxic moiety.

In some aspects, the dipeptide is Val-Cit. In some aspects, the cytotoxic moiety is exatecan.

In some aspects, the linker L is *-A-W₂—B₁-#, having the structure:

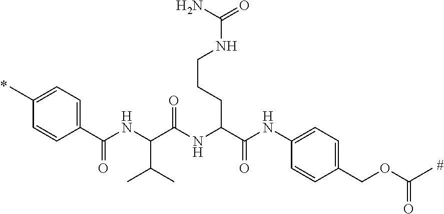

wherein * indicates the attachment point to the Y and # indicates the attachment point to the cytotoxic moiety.

In some aspects, C is a cytotoxic moiety. The term “cytotoxic moiety” or sometimes “payload,” refer to a chemical or biochemical moiety that is conjugated to the anti-FucGM1 antibody via a linker.

In some aspects, the n in the ADC of the formula (I) is 7 or 8. In some aspects, the n in the ADC is 5 or 6. In some aspects, the n in the ADC is 9 or 10. In some aspects, the n in the ADC is 7. In some aspects, the n in the ADC is 8.

In some aspects, in the formula (I), R¹ is a polyalkylene glycol unit having the structure:

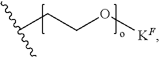

wherein: [symbol] indicates the position of the O;
K^{F} is H;
o is an integer ranging from 8 to 30;
L is a linker having the following structure:

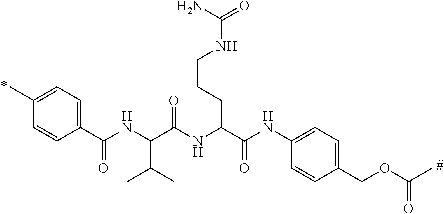

wherein * indicates the attachment point to the Y and # indicates the attachment point to the cytotoxic moiety;
C is exatecan;
m is 1; and
n ranges from 4-8.

In some aspects, in the formula (I), AB is an anti-FucGM1 antibody or an antigen-binding portion thereof discloses herein, Y is NH; n ranges from 5-10; R¹ is a polyalkylene glycol unit having the structure:

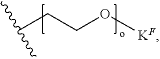

wherein: [symbol] indicates the position of the O; K^{F} is H; and
is an integer ranging from 8 to 30; L is a linker having the following structure:

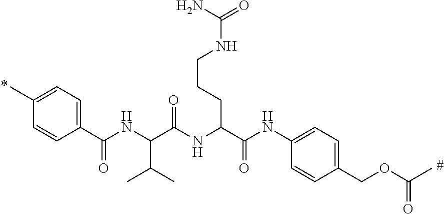

wherein * indicates the attachment point to Y and # indicates the attachment point to the cytotoxic moiety; C is exatecan; and m is 1.

In some aspects, o is an integer ranging from 8 to 28. In some aspects, o is an integer ranging from 8 to 16. In some aspects, o is 10, 11, 12, 13 or 14. In some aspects, o is an integer ranging from 20 to 28. In some aspects, o is 22, 23, 24, 25, or 26.

In some aspects, n ranges from 4 to 8. In some aspects, n ranges from 6 to 8.

In some aspects, n is an integer ranging from 2 to 10.

In some aspects, the VH comprises the amino acid sequence set forth in SEQ ID NO: 1 and the VL comprises the amino acid sequence set forth in SEQ ID NO: 2.

In some aspects, the anti-fucosyl-GM1 antibody or antigen binding portion thereof cross-competes with BMS-986012 for binding to fucosyl-GM1, and further wherein BMS-986012 comprises heavy and light chain variable regions comprising the amino acid sequences set forth in SEQ ID NOs: 1 and 2, respectively.

In some aspects, the anti-fucosyl-GM1 antibody, or antigen-binding portion thereof, comprises a heavy chain variable region comprising:
i. CDRH1 comprising the sequence set forth in SEQ ID NO: 5;
ii. CDRH2 comprising the sequence set forth in SEQ ID NO: 6; and
iii. CDRH3 comprising the sequence set forth in SEQ ID NO: 7

In some aspects, the anti-fucosyl-GM1 antibody, or antigen binding portion thereof, comprises a light chain variable region comprising:
i. CDRL1 comprising the sequence of SEQ ID NO: 8;
ii. CDRL2 comprising the sequence of SEQ ID NO: 9; and
iii. CDRL3 comprising the sequence of SEQ ID NO: 10.

In some aspects, the anti-fucosyl-GM1 antibody, or antigen binding portion thereof, comprises:
a. a heavy chain variable region comprising:
i. CDRH1 comprising the sequence set forth in SEQ ID NO: 5;
ii. CDRH2 comprising the sequence set forth in SEQ ID NO: 6; and
iii. CDRH3 comprising the sequence set forth in SEQ ID NO: 7; and;
b. a light chain variable region comprising:
i. CDRL1 comprising the sequence set forth in SEQ ID NO: 8;
ii. CDRL2 comprising the sequence set forth in SEQ ID NO: 9; and iii. CDRL3 comprising the sequence set forth in SEQ ID NO: 10.

In some aspects, the heavy chain variable region comprising the sequence set forth in SEQ ID NO: 1. In some aspects, the light chain variable region comprising the sequence set forth in SEQ ID NO: 2.

In some aspects, the anti-fucosyl-GM1 antibody, or antigen binding portion thereof, comprises a heavy chain variable region that is at least 80%, at least 85%, at least 90%, at least 95%, at least 96%, at least 97%, at least 98%, or at least 99% identical to the amino acid sequence set forth in SEQ ID NO: 1. In some aspects, the anti-fucosyl-GM1 antibody, or antigen binding portion thereof, comprises a light chain variable region that is at least 80%, at least 85%, at least 90%, at least 95%, at least 96%, at least 97%, at least 98%, or at least 99% identical to the amino acid sequence set forth in SEQ ID NO: 2.

In some aspects, the heavy chain variable region comprises the sequence set forth in SEQ ID NO: 1; and the light chain variable region comprises the sequence set forth in SEQ ID NO: 2.

In some aspects, the anti-fucosyl-GM1 antibody, or antigen binding portion thereof, comprises a heavy chain comprising the sequence set forth in SEQ ID NO: 3. In some aspects, the anti-fucosyl-GM1 antibody, or antigen binding portion thereof, comprises a light chain comprising the sequence set forth in SEQ ID NO: 4.

In some aspects, the anti-fucosyl-GM1 antibody, or antigen binding portion thereof, comprises:
a. a heavy chain comprising the sequence set forth in SEQ ID NO: 3; and
b. a light chain comprising the sequence set forth in SEQ ID NO: 4.

In some aspects, the anti-fucosyl-GM1 antibody is fucosylated. In some aspects, the anti-fucosyl-GM1 antibody is non-fucosylated. In some aspects, the anti-fucosyl-GM1 antibody comprises an IgG1 constant domain.

In some aspects, the ADC of the present disclosure has the formula (II):

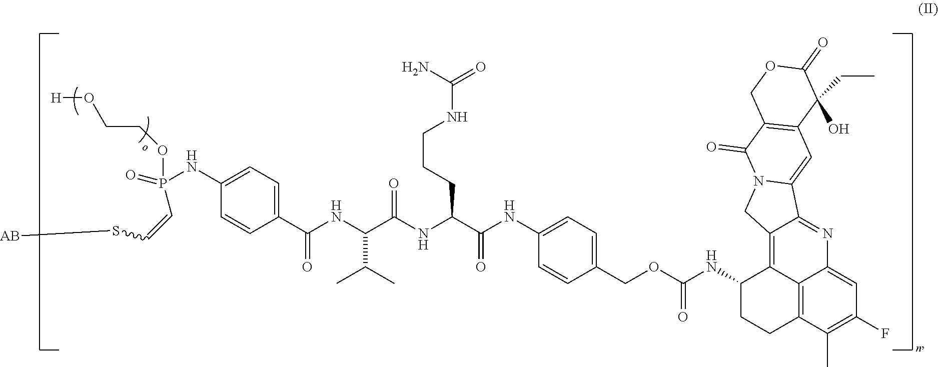

or a pharmaceutically acceptable salt, a stereoisomer, or a solvate thereof, wherein
[symbol] indicates that the configuration of the double bond may be E or Z o is an integer from 8 to 30;
n ranges from 4 to 9; and
AB is an antic-Fucosyl-GM1 antibody or antigen binding portion thereof disclosed herein. In some aspects, the linker is present as a mixture of the E and Z isomers.

In some aspects, o is an integer of 8, 9, 10, 11, 12, 13, 14, 15, 16, 17, 18, 19, 20, 21, 22, 23, 24, 25, 26, 27, 28, 29, or 30. In some aspects, o is an integer between 10 to 30. In some aspects, o is an integer between 8 and 16. In some aspects, o is 10, 11, 12, 13 or 14. In some aspects, o is an integer between 20 to 30, e.g., 24. In some aspects, o is an integer between 20 to 28. In some aspects, o is an integer between 20 and 25, e.g., 24. In some aspects, o is 22, 23, 24, 25, or 26. In some aspects, o is an integer of 24. In some aspects, o is 25. In some aspects, o is 23. In some aspects, o is 22. In some aspects, o is 21. In some aspects, o is 20. In some aspects, o is an integer between 26 and 30. In some aspects, o is 26. In some aspects, o is 27. In some aspects, o is 28. In some aspects o is 29. In some aspects, o is 30. In some aspects o is an integer between 8 and 19. In some aspects, o is 8. In some aspects, o is 9. In some aspects, o is 10. In some aspects o is 11. In some aspects o is 12. In some aspects o is 13. In some aspects, o is 14.

In some aspects, o is 15. In some aspects o is 16. In some aspects o is 17. In some aspects, o is 18. In some aspects, o is 19.

In some aspects, n ranges from 4 to 8. In some aspects, n ranges from 6 to 8. In some aspects, n is 4, 5, 6, 7, 8, or 9. In some aspects, n is 4. In some aspects, n is 5. In some aspects, n is 6. In some aspects, n is 7. In some aspects, n is 8. In some aspects, n is 9.

In some aspects, o is 24 and n is 4. In some aspects, o is 24 and n is 8.

In some aspects, the AB comprises a VH and a VL, which the VH comprises the amino acid sequence set forth in SEQ ID NO: 1 and the VL comprise the amino acid sequence set forth in SEQ ID NO: 2, respectively.

In some aspects, the anti-fucosyl-GM1 antibody, or antigen binding portion thereof, comprises a heavy chain comprising the sequence set forth as SEQ ID NO: 3. In some aspects, the anti-fucosyl-GM1 antibody, or antigen binding portion thereof, comprises a light chain comprising the sequence set forth as SEQ ID NO: 4. In some aspects, the anti-fucosyl-GM1 antibody, or antigen binding portion thereof, comprises a heavy chain comprising the sequence set forth as SEQ ID NO: 3; and a light chain comprising the sequence set forth as SEQ ID NO: 4.

In some aspects, the ADC of the present disclosure has the formula (II):

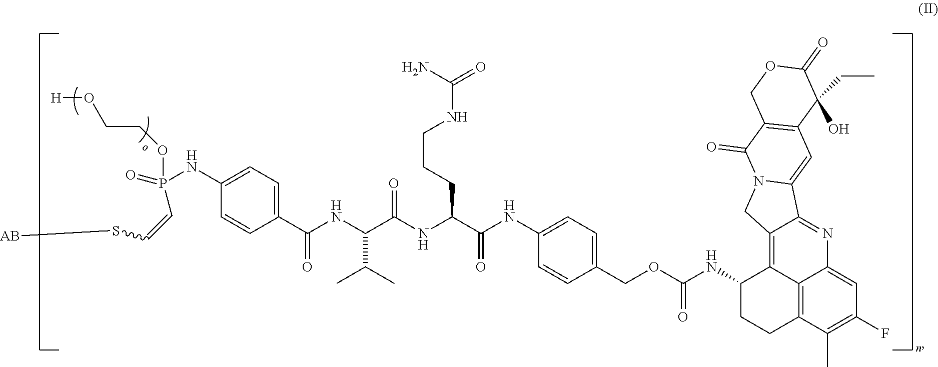

or a pharmaceutically acceptable salt, a stereoisomer or a solvate thereof; wherein n ranges from about 4 to 9; o is an integer ranging from 10 to 30; and AB is an anti-fucosyl-GM1 antibody or antigen binding portion thereof comprising a VH and a VL, which the VH comprises the amino acid sequence set forth in SEQ ID NO: 1 and the VL comprise the amino acid sequence set forth in SEQ ID NO: 2, respectively.

In some aspects, o is an integer of 8, 9, 10, 11, 12, 13, 14, 15, 16, 17, 18, 19, 20, 21, 22, 23, 24, 25, 26, 27, 28, 29, or 30. In some aspects, o is an integer between 10 to 30. In some aspects, o is an integer between 8 and 16. In some aspects, o is 10, 11, 12, 13 or 14. In some aspects, o is an integer between 20 to 30, e.g., 24. In some aspects, o is an integer between 20 to 28. In some aspects, o is an integer between 20 and 25, e.g., 24. In some aspects, o is 22, 23, 24, 25, or 26. In some aspects, o is an integer of 24. In some aspects, o is 25. In some aspects, o is 23. In some aspects, o is 22. In some aspects, o is 21. In some aspects, o is 20. In some aspects, o is an integer between 26 and 30. In some aspects, o is 26. In some aspects o is 27. In some aspects, o is 28. In some aspects o is 29. In some aspects, o is 30. In some aspects o is an integer between 8 and 19. In some aspects, o is 8. In some aspects, o is 9. In some aspects, o is 10. In some aspects o is 11. In some aspects o is 12. In some aspects o is 13. In some aspects, o is 14. In some aspects, o is 15. In some aspects o is 16. In some aspects o is 17. In some aspects, o is 18. In some aspects, o is 19.

In some aspects, n ranges from 4 to 8. In some aspects, n ranges from 6 to 8. In some aspects, n is 4, 5, 6, 7, 8, or 9. In some aspects, n is 4. In some aspects, n is 5. In some aspects, n is 6. In some aspects, n is 7. In some aspects, n is 8. In some aspects, n is 9.

In some aspects, o is 24 and n is 4. In some aspects, o is 24 and n is 8.

In some aspects, the anti-fucosyl-GM1 antibody, or antigen binding portion thereof, comprises a heavy chain comprising the sequence set forth as SEQ ID NO: 3. In some aspects, the anti-fucosyl-GM1 antibody, or antigen binding portion thereof, comprises a light chain comprising the sequence set forth as SEQ ID NO: 4. In some aspects, the anti-fucosyl-GM1 antibody, or antigen binding portion thereof, comprises a heavy chain comprising the sequence set forth as SEQ ID NO: 3; and a light chain comprising the sequence set forth as SEQ ID NO: 4.

The present disclosure also provides a compound of the formula (III):

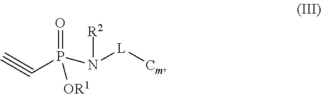

or a pharmaceutically acceptable salt, a stereoisomer, or a solvate thereof, wherein R¹, R², L, C, and m are as defined in the formula (I).

In some aspects, the present disclosure provides a method of preparing an ADC of the formula (I), comprising reacting a compound of the formula (III) with a thiol containing compound, AB-(SH)n, wherein AB is the anti-FucGM1 antibody or antigen binding portion thereof disclosed herein, n ranges from 1 and 10, to yield the ADC of the formula (I):

In some aspects, the compounds of the formula (III) have the structure:

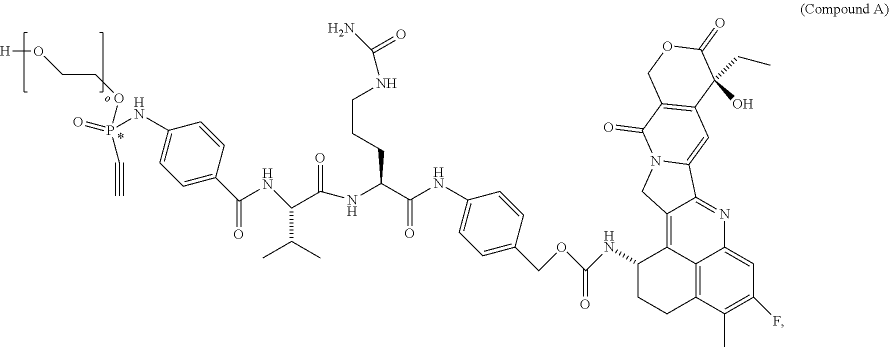

or a pharmaceutically acceptable salt a stereoisomer, or a solvate thereof, wherein o is an integer ranging from 8-25, e.g., 24, * represents a chiral center. All stereoisomers of Compound A are contemplated for the synthesizing the ADCs disclosed herein.

In some aspects, the present disclosure provides a method of preparing an ADC of the formula (II), comprising reacting Compound A with a thiol containing compound, AB-(SH)ₙ, wherein AB is the anti-FucGM1 antibody or antigen binding portion thereof disclosed herein, to obtain the ADC of the formula (II):

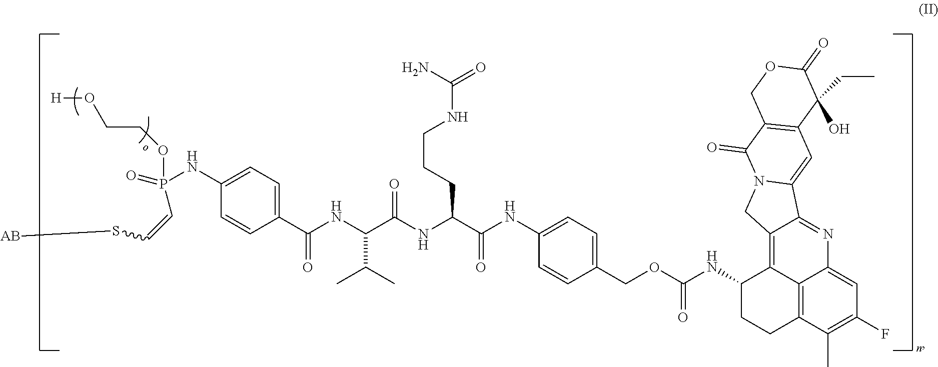

or a pharmaceutically acceptable salt, a stereoisomer, or a solvate thereof, wherein o and n are as defined above.

Methods of selective bioconjugation reaction of ethynylphosphonamideates with cysteine containing compounds have been described in WO2018041985A1 (published Mar. 8, 2018), WO2019170710A2 (published Sep. 12, 2019), WO2022223783A1 (published Oct. 27, 2022), WO2023083900A1 (published May 19, 2023), WO2023083919A1 (published May 19, 2023), each of which is incorporated herein by reference.

The number of cytotoxic moieties linked to the antigen binding moiety of a FucGM1-ADC (drug-to-antibody ratio: DAR) can vary and will be limited only by the number of available attachments sites on the antigen binding moiety and the number of agents linked to a single linker.

The DAR value can vary with the nature of the antigen binding moiety (e.g., any antibody or the antigen-binding portion thereof described herein) and the drug used along with the experimental conditions used for the conjugation (DAR, reaction time, nature of the solvents and/or cosolvents). Thus, the contact between the antibody and the drug may in an ADC lead to a mixture comprising several conjugates differing from one another by different drug-to-antibody ratios and may further include free antibodies and/or aggregates. The DAR that is determined is thus a mean value. DARs may be analyzed by UV spectrometry, monomer content may be analyzed by SEC-HPLC, and free drug content may be analyzed by RP-HPLC.

In some aspects, a linker will link a single cytotoxic moiety to the antigen binding moiety (e.g., any antibody or the antigen-binding portion thereof described herein) of a conjugate. In some aspects where the conjugate include more than one cytotoxic moiety, each moiety may be the same or different. As long as the conjugate does not exhibit unacceptable levels of aggregation under the conditions of use and/or storage, conjugates with DARs of twenty, or even higher, are contemplated. In some aspects, the conjugates described herein may have a DAR in the range of about 1-10, 2-10, 1-8, 2-8, 1-6, 2-6, 1-4, or 2-4. In some specific aspects, the conjugate may have a DAR of 2, 3, 4 or 5. In some aspects, the DAR is 6. In some aspects, the DAR is 7. In some aspects, the DAR is 8. In some aspects, the DAR is 9. In some aspects, the DAR is 6 or 7. In some aspects, the DAR is 7, 7.5 or 8. In some aspects, the DAR is 7-8.

In some aspects, an ADC of the present disclosure has the following structure:

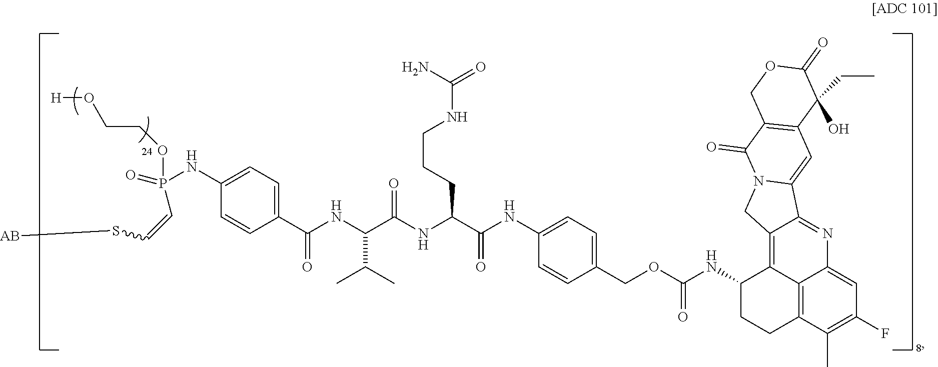

or a pharmaceutically acceptable salt, a stereoisomer, or a solvate thereof, wherein [symbol] represents that the configuration of the double bond may be E or Z; AB is the antibody, or antigen binding portion thereof that binds FucGM1, comprising a VH and a VL, which comprise the amino acid sequences set forth in SEQ ID NOs: 1 and 2, respectively.

The ADC 101 of the structure:

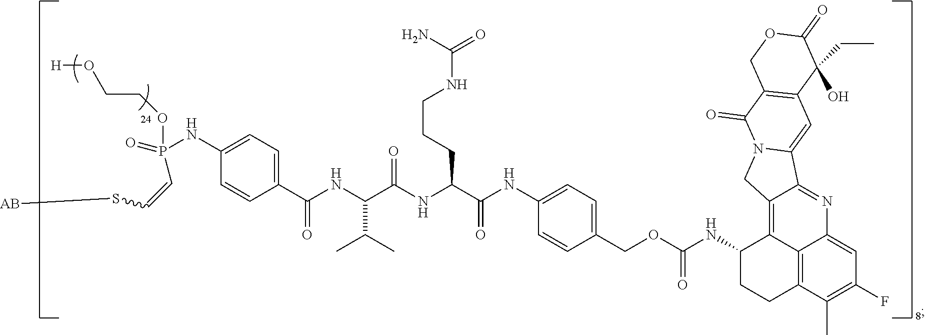

is also understood to be identical to ADC 101′ having the formula:

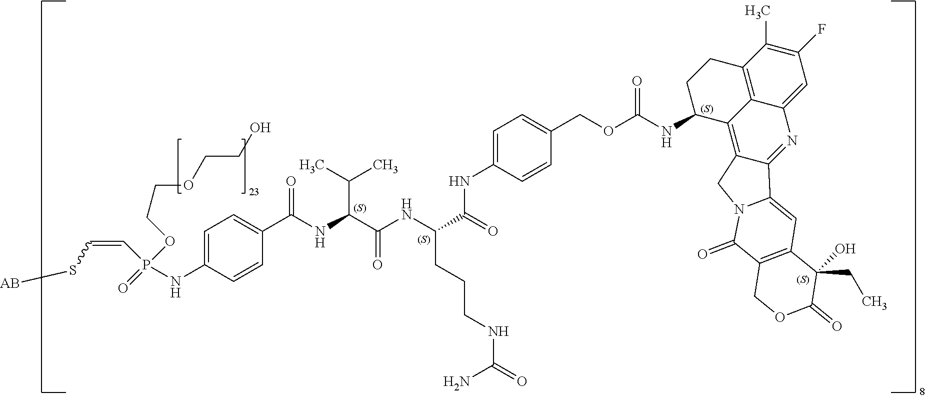

[ADC 101′], wherein [symbol] and AB have the same definition of those in ADC 101.

[symbol]

As the two structures are presumed to be identical, the two representations are interchangeable. Either structure can be used to represent the same structure. In some aspects, the disclosure provides ADC 101, or a pharmaceutically acceptable salt thereof, wherein AB is the anti-FucGM1 antibody, or antigen binding portion thereof, comprising a heavy chain and a light chain, which comprise the amino acid sequence set forth in SEQ ID NO: 3 and the amino acid sequence set forth in SEQ ID NO: 4, respectively.

ADCs can also be used to modify a given biological response, where the cytotoxic moiety should not be construed as limited to classical chemical therapeutic agents. For example, the cytotoxic moiety may be a protein or polypeptide possessing a desired biological activity (e.g., lymphokines, tumor necrosis factor, IFNγ, growth factors). Techniques for conjugating toxins or therapeutic moieties to antibodies are known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

In some aspects, the antibody is a human, humanized, or chimeric antibody. In some aspects, the antigen binding portion thereof comprises a Fab, Fab′, (Fab′)2, Fv, or scFv fragment.

### IV. Pharmaceutical Compositions

Also provided herein are pharmaceutical compositions comprising an ADC as disclosed herein and a carrier (e.g., pharmaceutically acceptable carrier). Such compositions are useful for various therapeutic applications, such as cancer treatment.

In some aspects, the pharmaceutical compositions may further include other compounds, drugs, and/or agents for various therapeutic applications. Such compounds, drugs, and/or agents can include, for example, an anti-cancer agent, a chemotherapeutic agent, an immunosuppressive agent, an immunostimulatory agent, an immune checkpoint inhibitor, and/or an anti-inflammatory agent. Exemplary compounds, drugs, and agents that can be formulated together or separately with the ADC described in the next section.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein may also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. Except insofar as any media or agent is incompatible with the active compound, use thereof in the pharmaceutical compositions described herein is contemplated. A pharmaceutical composition may comprise a preservative or may be devoid of a preservative. Supplementary active compounds can be incorporated into the compositions.

A composition described herein can be administered via one or more routes of administration using one or more of a variety of methods. The route and/or mode of administration can vary depending upon the desired results. Routes of administration for the ADC described herein include e.g., intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an ADC described herein can be administered via a non-parenteral route, such as a topical, epidermal or mucosal route of administration, for example, intranasally, orally, vaginally, rectally, sublingually or topically.

### V. Uses and Methods

The ADC described herein have numerous in vitro and in vivo utilities as described herein.

### Cancer Treatment

Certain aspects of the disclosure are directed to methods of treating cancer comprising administering to a subject in need thereof an ADC as disclosed herein in an effective amount so that the growth of a cancerous tumor is inhibited or reduced and/or that regression and/or that prolonged survival is achieved. In some aspects, the cancer is associated with expression of FucGM1. For example, the FucGM1expression in the cancer is higher than the FucGM1 expression from a tissue sample from a healthy subject. In certain embodiments, the subject has been diagnosed with having a solid tumor cancer.

Certain aspects of the disclosure are directed to an in vitro method of killing cells expressing FucGM1 comprising contacting a sample comprising the cells with an ADC as disclosed herein. In some aspects, the cells expressing FucGM1 comprises cancer cells.

In some aspects, the ADC described herein may be administered in combination with additional cytotoxic or therapeutic agent(s), for example as described herein.

Cancers that express FucGM1 whose growth may be inhibited using the ADC described herein include, but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer (e.g. estrogen-receptor positive breast cancer HER2-positive breast cancer; triple negative breast cancer); cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; liver cancer (e.g., hepatocellular carcinoma; hepatoma); intra-epithelial neoplasm; kidney or renal cancer; larynx cancer; leukemia; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblasts leukemia; and post-transplant lymphoproliferative disorder (PTLD), as well as abnormal vascular proliferation associated with phakomatoses, edema (such as that associated with brain tumors), tumors of primitive origins and Meigs' syndrome.

Additional cancers which express FucGM1 and can be treated using the ADC described herein include metastatic pancreatic cancer, metastatic adenocarcinoma of the pancreas, stomach cancer, fibrotic cancer, glioma, malignant glioma, diffuse intrinsic pontine glioma, recurrent childhood brain neoplasm renal cell carcinoma, clear-cell metastatic renal cell carcinoma, metastatic castration resistant prostate cancer, stage IV prostate cancer, metastatic melanoma, malignant melanoma, recurrent melanoma of the skin, melanoma brain metastases, malignant melanoma of head and neck, squamous cell non-small cell lung cancer, metastatic breast cancer, follicular lymphoma, advanced B-cell NHL, HL including diffuse large B-cell lymphoma (DLBCL), multiple myeloma, chronic myeloid leukemia, adult acute myeloid leukemia in remission, adult acute myeloid leukemia with Inv(16)(p13.1q22), CBFB-MYH11, adult acute myeloid leukemia with t(16:16) (p13.1:q22), CBFB-MYH11, adult acute myeloid leukemia with t(8:21)(d22:q22), RUNX1-RUNX1T1, adult acute myeloid leukemia with t(9:11)(p22:q23), MLLT3-MLL, adult acute promyelocytic leukemia with tO15:17)(q22:ql2), PML-RARA, alkylating agent-related acute myeloid leukemia, Richter's syndrome, adult glioblastoma, adult gliosarcoma, recurrent glioblastoma, recurrent childhood rhabdomyosarcoma, recurrent Ewing sarcoma/peripheral primitive neuroectodermal tumor, recurrent neuroblastoma, recurrent osteosarcoma, colorectal cancer, MSI positive colorectal cancer, MSI negative colorectal cancer, nasopharyngeal nonkeratinizing carcinoma, recurrent nasopharyngeal undifferentiated carcinoma, cervical adenocarcinoma, cervical adenosquamous carcinoma; cervical squamous cell carcinoma, recurrent cervical carcinoma, anal canal squamous cell carcinoma, metastatic anal canal carcinoma, recurrent anal canal carcinoma, recurrent head and neck cancer, squamous cell of head and neck, head and neck squamous cell carcinoma (HNSCC), ovarian carcinoma, colon cancer, advanced GI cancer, gastric adenocarcinoma, gastroesophageal junction adenocarcinoma, bone neoplasms, soft tissue sarcoma, bone sarcoma, thymic carcinoma, urothelial carcinoma, Merkel cell carcinoma, recurrent Merkel cell carcinoma, mycosis fungoides, Sezary syndrome, neuroendocrine cancer, nasopharyngeal cancer, basal cell skin cancer, squamous cell skin cancer, dermatofibrosarcoma trotuberans, glioma, mesothelioma, myelodysplastic syndromes (MDS), myelofibrosis (MF), myeloproliferative neoplasms, and acute myeloid leukemia (AML).

Cancers may be, e.g., metastatic or primary cancers; desmoplastic or non-desmoplastic cancers; or recurrent cancers.

In some aspects, the cancer comprises colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, myelodysplastic syndromes, or any combination thereof.

In some aspects, the lung cancer is small cell lung cancer (SCLC).

In some aspects, the cancer is associated with fibrosis. In some aspects, the cancer is associated with infiltration of CD4+ regulatory T cells. In some aspects, the cancer is associated with infiltration of CD8+ regulatory T cells. In some aspects, the cancer is associate with infiltration of regulatory B cells. In some aspects, the cancer is associated with infiltration of myeloid-derived suppressor cells. In some aspects, the cancer is associated with infiltration of tumor-associated macrophages. In some aspects, the cancer is associated with infiltration of innate lymphoid cells. In some aspects, the cancer is associated with infiltration of cancer-associated fibroblasts. In some aspects, the cancer is associated with a radiation-related increase in the above cell types.

In some aspects, the ADCs described herein are used to treat myelodysplastic syndromes (MDSs). MDSs are a diverse group of malignant disorders marked by bone marrow failure due to defective hematopoiesis and production of dysplastic cells. TGF-β is a primary driver in MDS (Geyh et al., Haematologica 2018; 103:1462-71) and agents that inhibit the function of TGF-β have been proposed as therapeutics (Mies et al., Curr Hematol Malig Rep 2016; 11:416-24). Furthermore, MDSCs are known to be dysregulated in MDS (Chen et al., JCI 2013; 123:4595-611) and agents that reduce MDSC levels in the bone marrow are potential therapeutics.

In some aspects, the cancer is resistant to checkpoint inhibitor(s). In some aspects, the cancer is intrinsically refractory or resistant (e.g., resistant to a PD-1 pathway inhibitor, PD-1 pathway inhibitor, or CTLA-4 pathway inhibitor). In some aspects, the resistance or refractory state of the cancer is acquired. In some aspects, the ADC described herein can be used in combination with checkpoint inhibitors to overcome resistance of the cancer to the checkpoint inhibitors. In some aspects, the ADC described herein can be used to treat tumors with a mesenchymal and/or EMT signature together with checkpoint inhibitors in combination or sequentially with agents that induce a mesenchymal phenotype, such as MAPK pathway inhibitors.

In some aspects, the ADCs described herein are used to enhance the viability of immune cells ex vivo, e.g., in adoptive natural killer (NK) cell transfer. Accordingly, in some aspects, ADCs are used in combination with adoptively transferred NK cells to treat cancer. In some aspects, the ADC described herein are used to treat tumors with MHC loss or MHC down-regulation, as monotherapy or in combination with NK activating or enhancing treatment.

### Combination Therapy

Certain aspects of the disclosure are directed to methods of treating cancer comprising administering to a subject in need thereof an ADC as disclosed herein in combination with one or more additional therapies as described herein, in an effective amount so that the growth of a cancerous tumor is inhibited or reduced and/or that regression and/or that prolonged survival is achieved. In some aspects, the cancer is associated with expression of FucGM1. In some aspects, the one or more additional therapies comprise cytotoxic or therapeutic agent(s).

Certain aspects of the disclosure are directed to an in vitro method of killing cells expressing FucGM1 comprising contacting a sample comprising the cells with an ADC as disclosed herein in combination with additional one or more additional therapies as described herein. In some aspects, the cells expressing FucGM1 comprises cancer cells. In some aspects, the one or more additional therapies comprise cytotoxic or therapeutic agent(s).

The ADCs described herein can be used in combination with various treatments or agents (or in the context of a multispecific antibody or bifunctional partner) known in the art for the treatment of disease or condition (e.g., cancer), as described herein.

In some aspects, a method of treating cancer comprises administering to a subject in need thereof an effective amount of an ADC described herein in combination with another therapeutic agent (e.g., an additional therapy), such as a second antibody, a therapeutic protein or a small molecule drug. In some aspects, the therapeutic protein is a checkpoint inhibitor. In some aspects, the small molecule drug is a chemotherapeutic agent as described herein. In some aspects, the another therapeutic agent comprises an anti-cancer agent.

Suitable anti-cancer agents for use in combination therapy with the ADC described herein include, but are not limited to, surgery, chemotherapeutic agents, growth inhibitory agents, cytotoxic agents, radiotherapy and agents used in radiation therapy, anti-angiogenesis agents, apoptotic agents, anti-tubulin agents, and other agents to treat cancer. Combinations thereof are also specifically contemplated for the methods described herein. In some aspects, the one or more additional therapies comprises radiation therapy, chemotherapy, immune checkpoint inhibitor therapy, CAR-T therapy, immunosuppressive therapy, immunostimulatory therapy, cell therapy, a therapeutic agent, or any combination thereof. In some aspects, the immune checkpoint therapy comprises administering an immune checkpoint inhibitor.

In some aspects, the ADC is administered with an anti-cancer agent, such as an EGFR inhibitor; a HER2 inhibitor; a histone deacetylase inhibitor; a hormone; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a microtubule targeting drug; a topoisomerase poison drug; or a combination thereof.

In some aspects, the ADC is administered along with an immune checkpoint inhibitor. Exemplary immune checkpoint inhibitors include, but are not limited to, agents (e.g., antibodies) that bind to PD-1, PD-L1, PD-L2, LAG-3, CTLA4, TIGIT, ICOS, OX40, PVR, PVRIG, VISTA, TIM3, SIRPα, ILT2, ILT3, ILT4, or ILT5. In some aspects, the immune checkpoint inhibitor comprises an anti-PD-1 antibody, an anti-PD-L1 antibody, an anti-LAG-3 antibody, an anti-CTLA-4 antibody, an anti-TIGIT antibody, an anti-TIM3 antibody, or any combination thereof.

In certain embodiments, the immune checkpoint inhibitor is a composition comprising an anti-LAG-3 antibody and an anti-PD-1 antibody. See U.S. Pat. Nos. 9,505,839 and 11,236,163.

Any anti-PD-1 antibody can be used in combination with the ADC in the presently described methods. Various human monoclonal antibodies that bind specifically to PD-1 with high affinity have been disclosed in U.S. Pat. No. 8,008,449. In some aspects, the immune checkpoint inhibitor is an anti-PD1 antibody.

In some aspects, the anti-PD-1 antibody is pembrolizumab, nivolumab, cemiplimab, spartalizumab, camrelizumab, sintilimab, tislelizumab, toripalimab, dostarlimab, retifanlimab, pimivalimab, dostarlimab, serplulimab, zimberelimab, acrixolimab, MEDI-0680, AM-0001, STI-1110, AGEN2034, BCD-100, sasanlimab, BI 754091, or SSI-361.

In some aspects, the anti-PD-1 antibody used in combination with the ADC comprises the CDRH1, CDRH2, CDRH3, CDRL1, CDRL2, CDRL3, the VH and the VL, and/or the heavy and light chains of any of pembrolizumab, nivolumab, cemiplimab, spartalizumab, camrelizumab, sintilimab, tislelizumab, toripalimab, dostarlimab, retifanlimab, pimivalimab, MEDI-0680, GLS-010, AM-0001, STI-1110, AGEN2034, BCD-100, sasanlimab, BI 754091, or SSI-361.

In some aspects, the anti-PD-1 antibody used in combination with the ADC is selected from the group consisting of nivolumab (OPDIVO®; formerly designated 5C4, BMS-936558, MDX-1106, or ONO-4538), pembrolizumab (KEYTRUDA®; formerly designated lambrolizumab and MK-3475; see WO 2008/156712A1), PDR001 (see WO 2015/112900), MEDI-0680 (formerly designated AMP-514; see WO 2012/145493), REGN-2810 see WO 2015/112800), JS001 (see Liu and Wu, 2017), BGB-A317 (see WO 2015/035606 and US 2015/0079109), INCSHR1210 (SHR-1210; see WO 2015/085847; Liu and Wu, 2017), TSR-042 (ANB011; see WO 2014/179664), GLS-010 (WBP3055; see Liu and Wu, 2017), AM-0001 (see WO 2017/123557), STI-1110 (see WO 2014/194302), AGEN2034 (see WO 2017/040790), and MGD013 (see WO 2017/106061).

In some aspects, the anti-PD-1 antibody used in combination with the ADC is pembrolizumab (Merck; also known as KEYTRUDA®, lambrolizumab, and MK-3475; see, for example, WO 2008/156712). Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

In some aspects, the anti-PD-1 antibody used in combination with the ADC comprises nivolumab (also known as OPDIVO®, 5C4, BMS-936558, MDX-i 106, and ONO-4538). Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (see, for example, U.S. Pat. No. 8,008,449; Wang et al., 2014 Cancer Immunol Res. 2(9):846-56).

In some aspects, the anti-PD-1 antibody used in combination with the ADC is cemiplimab (Regeneron; also known as LIBTAYO or REGN-2810; see, for example, WO 2015/112800 and U.S. Pat. No. 9,987,500).

In some aspects, the anti-PD-1 antibody used in combination with the ADC is spartalizumab (Novartis; also known as PDR001; see, for example, WO 2015/112900 and U.S. Pat. No. 9,683,048).

In some aspects, the anti-PD-1 antibody used in combination with the ADC is camrelizumab (Jiangsu Hengrui Medicine; also known as SHR-1210 or INCSHR1210; see, for example, WO 2015/085847; Si-Yang Liu et al., J. Hematol. Oncol. 10:136 (2017)).

In some aspects, the anti-PD-1 antibody used in combination with the ADC is MEDI-0680 (AstraZeneca; also known as AMP-514; see, for example, WO 2012/145493). In some aspects, the anti-PD-1 antibody is pimivalimab (also known as JTX-4014; see, for example, Papadopoulos, et al., 2022, IOTECH, Vol. 16, Supplement 1, 100284). In some aspects, the anti-PD-1 antibody is toripalimab (TAIZHOU JUNSHI PHARMA; also known as JS001; see, for example, Si-Yang Liu et al., J. Hematol. Oncol. 10:136 (2017)). In some aspects, the anti-PD-1 antibody is tislelizumab (Beigene; also known as BGB-A317; see, for example, WO 2015/35606 and US 2015/0079109). In some aspects, the anti-PD-1 antibody is dostarlimab (Tesaro Biopharmaceutical; also known as ANB011 or TSR-042; see, for example, WO2014/179664). In some aspects, the anti-PD-1 antibody is GLS-010 (Wuxi/Harbin Gloria Pharmaceuticals; also known as WBP3055; see, for example, Si-Yang Liu et al., J. Hematol. Oncol. 10:136 (2017)). In some aspects, the anti-PD-1 antibody is AM-0001 (Armo BioSciences).

In some aspects, the anti-PD-1 antibody is STI-1110 (Sorrento Therapeutics; see, for example, WO 2014/194302). In some aspects, the anti-PD-1 antibody is AGEN2034 (Agenus; see, for example, WO 2017/040790). In some aspects, the anti-PD-1 antibody is retifanlimab (Macrogenics, also known as MGA012, AEX-1188, and INCMGA-00012; see, for example, WO 2017/19846). In some aspects, the anti-PD-1 antibody is BCD-100 (Biocad; see, for example, Kaplon et al., mAbs 10(2):183-203 (2018). In some aspects, the anti-PD-1 antibody is sintilimab (Innovent; also known as IBI308; see, for example, WO 2017/024465, WO 2017/025016, WO 2017/132825, and WO 2017/133540). In some aspects, the anti-PD-1 antibody is sasanlimab (Pfizer; also known as PF06801591; see, for example, US 2016/0159905). In some aspects, the anti-PD-1 antibody is BI 754091 (Boehringer Ingelheim; see, for example, Zettl M et al., Cancer. Res. (2018); 78(13 Suppl):Abstract 4558). In some aspects, the anti-PD-1 antibody is SSI-361 (Lyvgen Biopharma Holdings Limited, see, for example, US 2018/0346569).

Other anti-PD-1 monoclonal antibodies suitable for the methods of the present disclosure have been described in, for example, U.S. Pat. Nos. 6,808,710, 7,488,802, 8,168,757, 8,354,509, and 9,205,148, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Examples of anti-PD-L1 antibodies useful in combination with the ADC according to the methods of the present disclosure include the antibodies disclosed in U.S. Pat. No. 9,580,507. In some aspects, the anti-PD-L1 antibody is atezolizumab, durvalumab, avelumab, envafolimab, cosibelimab, BMS-936559, STI-1014, CX-072, LY3300054, FAZ053, CS-1001, SHR-1316, CBT-502, KN035, or BGB-A333.

In some aspects, the anti-PD-L1 antibody is BMS-936559 (also known as 12A4, MDX-1105; see, e.g., U.S. Pat. No. 7,943,743 and WO 2013/173223).

In some aspects, the anti-PD-L1 antibody is STI-1014 (Sorrento; see, for example, WO 2013/181634). STI-104 is designated H6 in U.S. Pat. No. 9,175,082. In some aspects, the anti-PD-L1 antibody is CX-072 (Cytomx; see, for example, WO 2016/149201). In some aspects, the anti-PD-L1 antibody is LY3300054 (Eli Lilly Co.; see, e.g., WO 2017/034916). In some aspects, the anti-PD-L1 antibody is FAZ053 (Novartis). In some aspects, the anti-PD-L1 antibody is CK-301 (Checkpoint Therapeutics; see, for example, Gorelik et al., AACR:Abstract 4606 (April 2016)). CK-301 is also referred to as cosibelimab. In some aspects, the anti-PD-L1 antibody is CS-1001. See, for example, Zhou et al., Journal of Clinical Oncology, Meeting Abstract, 2020 ASCO Annual Meeting I, Lung Cancer—Non-Small Cell Metastatic, e21687, and Zhang et al., Cancer Research, 2020, 80 (16_Supplement): 3260. In some aspects, the anti-PD-L1 antibody is SHR-1316. See, for example, Mu et al., Thorac Cancer, 2021 May; 12(9):1373-1381, and Wu et al., Anals of Oncology, Abstract, Vol. 33, Supplement 2, S72, April 2022. In some aspects, the anti-PD-L1 antibody is CBT-502 (also known as TQB2450; see, for example, Wei et al., Mol Cancer Ther (2018) 17 (1_Supplement): A200). In some aspects, the anti-PD-L1 antibody is KN035 (3D Med/Alphamab; also referred to as envafolimab; see, for example, Zhang et al., Cell Discov. 7:3 (March 2017) and Shimizu et al., Invest New Drugs, 2022 October; 40(5):1021-1031).

In some aspects, the anti-PD-L1 antibody is BGB-A333 (BeiGene; see, for example, Desai et al., JCO 36 (15suppl):TPS3113 (2018) and Desai et al., 2023, British Journal of Cancer 128, 1418-1428). In certain aspects, the PD-L1 antibody is atezolizumab. Atezolizumab is a fully humanized IgG1 monoclonal anti-PD-L1 antibody. Atezolizumab (Roche) is also known as TECENTRIQ®; MPDL3280A, RG7446. See, for example, U.S. Pat. No. 8,217,149 and Herbst et al. (2013) J. Clin. Oncol. 31 (suppl):3000). Atezolizumab is designated YW243.55S70 in U.S. Pat. No. 8,217,149. In certain aspects, the PD-L1 antibody is durvalumab. Durvalumab is a human IgG1 kappa monoclonal anti-PD-L1 antibody. Durvalumab (AstraZeneca) is also known as IMFINZI® or MEDI-4736. Durvalumab is designated 2.14H9OPT in U.S. Pat. No. 8,779,108. See, for example, WO 2011/066389. In certain aspects, the PD-L1 antibody is avelumab. Avelumab is a human IgG1 lambda monoclonal anti-PD-L1 antibody. Avelumab (Pfizer) is also known as BAVENCIO® or MSB0010718C. Avelumab is designated A09-246-2 in U.S. Pat. No. 9,624,298. See, for example, WO 2013/079174.

In some aspects, the anti-CTLA-4 antibody useful in combination with the ADC is tremelimumab, ipilimumab, botensilimab, BMS-986218, BMS-986288, BMS-986249, IBI310, MK-1308 (quavonlimab), AGEN-1884 (zalifrelimab), ONC-392, ADG116, or CS1002.

In some aspects, the anti-CTLA-4 antibody useful in combination with the ADC is MK-1308. MK-1308 is also known as quavonlimab. See, for example, Perets et al. 2021, Ann Oncol 32(3):395-403.

In some aspects, the anti-CTLA-4 antibody useful in combination with the ADC is AGEN-1884. AGEN-1884 is also known as zalifrelimab. See, for example, WO 2016/196237.

In some aspects, the anti-CTLA-4 antibody useful in combination with the ADC is tremelimumab. Tremelimumab, sold under the brand name IMJUDO®, is a fully human monoclonal antibody used for the treatment of hepatocellular carcinoma and non-small cell lung cancer. Tremelimumab (AstraZeneca) is also known as ticilimumab, CP-675,206; see WO 2000/037504 and Ribas, Update Cancer Ther. 2(3): 133-39 (2007)).

In some aspects, the anti-CTLA-4 antibody useful in combination with the ADC is ipilimumab. Ipilimumab (sold under the brand name YERVOY®, which was first approved for the treatment of metastatic melanoma, has since been approved for use in other cancers. Hoos et al. (2010) Semin. Oncol. 37:533; Hodi et al. (2010) N. Engl. J. Med. 363:711; Pardoll (2012) Nat. Immunol. 13(12):1129. In 2011, ipilimumab is a human antibody, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed. Ipilimumab is also known as MDX-010 and 10D1. See U.S. Pat. No. 6,984,720.

In some aspects, the anti-CTLA-4 antibody is an activatable anti-CTLA-4 antibody, such as an activatable anti-CTLA-4 antibody wherein the light chains of the antibody comprise a cleavable moiety and a masking moiety at the amino termini. The masking moiety interferes with binding to CTLA-4, but is preferentially released in the tumor microenvironment after cleavage of the cleavable moiety by proteases that are more prevalent and/or active in tumors than in peripheral tissues (see, in particular, WO 2018/085555). Such preferential cleavage in the tumor microenvironment enables full CTLA-4 blocking, promoting anti-tumor immune response, while minimizing CTLA-4 blockade in normal tissue, thereby reducing the risk of potential systemic toxicity of an anti-CTLA-4 antibody. In some aspects, the activatable anti-CTLA-4 antibody is an activatable form of ipilimumab, such as an antibody comprising light chains modified to comprise a masking moiety and a cleavable moiety, as disclosed, for example, in WO 2018/085555. An example of an activatable anti-CTLA-4 antibody that has entered human clinical trials is BMS-986249 (NCT03369223: “A Study of BMS-986249 Alone and in Combination with Nivolumab in Advanced Solid Tumors”). In some aspects, the anti-CTLA-4 antibody is BMS-986249.

In some aspects, the anti-CTLA-4 antibody shows an enhanced Fcγ receptor (CD16) binding. Whether an anti-CTLA-4 antibody shows an enhanced Fcγ receptor binding is assessed by comparison with the Fcγ receptor binding of ipilimumab. Anti-CTLA-4 antibodies with enhanced Fcγ receptor (CD16) binding have been proposed as therapeutic agents for treatment of cancer through depletion of Treg cells. See, in particular, WO 2014/089113. In some aspects, the anti-CTLA-4 antibody shows an Fcγ receptor (CD16) binding that is at least two-fold enhanced when compared to the Fcγ receptor binding of ipilimumab.

Examples of anti-CTLA-4 antibodies that show enhanced Fcγ receptor (i.e., FcγRIIIA or CD16) binding are nonfucosylated anti-CTLA-4 antibodies. In some aspects, the anti-CTLA-4 antibody is a nonfucosylated anti-CTLA-4 antibody. Non-fucosylated anti-CTLA-4 antibodies lack fucose residues in its N-linked glycans. In some aspects, the non-fucosylated anti-CTLA-4 antibody is produced by expressing the chains of the antibody in a mammalian cell under conditions that prevent fucosylation, including but not limited to use of mammalian cells with genetic modifications preventing fucosylation, or growth of the cells expressing the antibody in medium containing one or more chemical compounds that inhibit fucosylation. In some aspects, the genetic modification that prevents fucosylation is inactivation, e.g. knock-out, of the FUT8 gene. In some aspects, the anti-CTLA-4 antibody is a hypofucosylated anti-CTLA-4 antibody.

An exemplary nonfucosylated anti-CTLA-4 antibody that has entered human clinical trials is BMS-986218 (e.g., NCT03110107: “First-In-Human Study of Monoclonal Antibody BMS-986218 by Itself and in Combination with Nivolumab in Participants with Advanced Solid Tumors”). BMS-986218 is a nonfucosylated antibody developed to increase the effects of CTLA-4 blockade by enhancing binding to Fc7 receptor, thus promoting APC-mediated T cell priming. In some aspects, the anti-CTLA-4 antibody is BMS-986218. See, for example, PCT/US18/19868.

In some aspects, the Fc region of the anti-CTLA-4 antibody contains amino acid substitutions in the antibody constant region to enhance binding to activating Fc7 receptors. Exemplary substitutions are G236A, S239D, A330L and 1332E (all residue numbering per the EU numbering system). In some aspects, the anti-CTLA-4 antibody comprises a human IgG1 constant domain with S239D, A330L and 1332E mutations.

In some aspects, the anti-CTLA-4 antibody is an activatable and nonfucosylated anti-CTLA-4 antibody.

Human monoclonal antibodies that bind specifically to CTLA-4 with high affinity that are suitable for the methods of the present disclosure have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety.

In some aspects, the anti-LAG-3 antibody useful in combination with the FucGM1 targeting agent according to the methods of the present disclosure is relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3(0414), aLAG-3(0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501

In some aspects, the anti-LAG-3 antibody useful in combination with the FucGM1 targeting agent comprises the CDRH1, CDRH2, CDRH3, CDRL1, CDRL2, CDRL3, the VH and the VL, and/or the heavy and light chains of any of relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3(0414), aLAG-3(0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, or ABL501.

In some aspects, the anti-LAG-3 antibody useful in combination with the ADC comprises relatlimab (BMS-986016). In some aspects, the anti-LAG-3 antibody comprises IMP731 (H5L7BW). In some aspects, the anti-LAG-3 antibody comprises MK4280 (28G-10, favezelimab). MK-4280 (28G-10, favezelimab) described in WO2016028672 and U.S. Publication No. 2020/0055938. In some aspects, the anti-LAG-3 antibody comprises REGN3767 (fianlimab). REGN3767 (fianlimab) is described, for example, in Burova E, et al., J. Immunother. Cancer (2016); 4(Supp. 1):P195 and U.S. Pat. No. 10,358,495. In some aspects, the anti-LAG-3 antibody comprises GSK2831781. In some aspects, the anti-LAG-3 antibody comprises humanized BAP050. Humanized BAP050 is described, for example, in WO2017/019894. In some aspects, the anti-LAG-3 antibody comprises IMP-701 (LAG525, ieramilimab) IMP-701 (LAG525; ieramilimab) is described, for example, in U.S. Pat. No. 10,711,060 and U.S. Publ. No. 2020/0172617. In some aspects, the anti-LAG-3 antibody comprises aLAG-3(0414). In some aspects, the anti-LAG-3 antibody comprises aLAG-3(0416). In some aspects, the anti-LAG-3 antibody comprises Sym022. In some aspects, the anti-LAG-3 antibody comprises TSR-033. In some aspects, the anti-LAG-3 antibody comprises TSR-075. In some aspects, the anti-LAG-3 antibody comprises XmAb841 (XmAb22841). In some aspects, the anti-LAG-3 antibody comprises MGD013 (tebotelimab). In some aspects, the anti-LAG-3 antibody comprises BI754111. In some aspects, the anti-LAG-3 antibody comprises FS118. In some aspects, the anti-LAG-3 antibody comprises P 13B02-30. In some aspects, the anti-LAG-3 antibody comprises AVA-017. In some aspects, the anti-LAG-3 antibody comprises 25F7. 25F7 is described, for example, in U.S. Publ. No. 2011/0150892. In some aspects, the anti-LAG-3 antibody comprises AGEN1746. In some aspects, the anti-LAG-3 antibody comprises RO7247669. In some aspects, the anti-LAG-3 antibody comprises INCAGN02385. In some aspects, the anti-LAG-3 antibody comprises IBI-110. In some aspects, the anti-LAG-3 antibody comprises EMB-02. In some aspects, the anti-LAG-3 comprises IBI-323. In some aspects, the anti-LAG-3 antibody comprises LBL-007. In some aspects, the anti-LAG-3 antibody comprises ABL501.

In general, any anti-LAG-3 antibody useful in combination with the ADC can be used. Antibodies that bind to LAG-3 have been disclosed in Int'l Publ. No. WO/2015/042246 and U.S. Publ. Nos. 2014/0093511 and 2011/0150892, each of which is incorporated by reference herein in its entirety. Disclosure relating to the anti-LAG-3 antibodies described herein and other anti-LAG-3 antibodies useful in the methods of the present disclosure can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

In some aspects, the ADC described herein may also be administered with a standard of care treatment, or another treatment, such as radiation, surgery, or chemotherapy. The ADC may be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells.

These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

### VI. Kits

Also provided are kits comprising an ADC described herein and instructions for use.

In some aspects, the kits comprise the ADC in unit dosage form, such as in a single dose vial or a single dose pre-loaded syringe, optionally contained in a single vial or container, along with e.g., instructions for use in treating a cancer as described herein.

The present disclosure is further illustrated by the following examples, which should not be construed as further limiting. The contents of all figures and all references, Genbank sequences, issued patents, and published patent applications cited throughout this disclosure are expressly incorporated herein by reference.

### VII. Examples

Commercially available reagents referred to in the Examples below were used according to manufacturer's instructions unless otherwise indicated. Unless otherwise noted, the present disclosure uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

The following Examples describe the isolation and characterization of anti-FucGM1 monoclonal antibodies. The CDR sequences, variable region sequences, and full-length heavy and light chain sequences of anti-FucGM1 antibodies are provided below, for example in Table 3.

### EXAMPLES

### Example 1. Production of Nonfucosylated HuMAb that Binds FucGM1

Antibodies with reduced amounts of fucosyl residues have been demonstrated to increase the ADCC ability of the antibody. In this example, an anti-Fucosyl GM1 HuMAb MBN-1 was produced that was lacking in fucosyl residues for enhanced ADCC activity.

The CHO cell line Ms704-PF, which lacked the fucosyltransferase gene, FUT 8 (Biowa, Inc., Princeton, NJ) was electroporated with a vector which expresses the heavy and light chains of an anti-Fucosyl GM1 HuMAb MBN-001 shown below in Table 3.

Drug-resistant clones are selected by growth in Ex-Cell 325-PF CHO media (JRN Biosciences, Lenexa, KS) with 6 mM L-glutamine and 500 μg/ml G418 (Invitrogen, Carlsbad, CA). Clones are screened for IgG expression by standard ELISA assay. Full antibodies were generated with an IgG1 effective Fc.

TABLE 3
SUMMARY OF SEQUENCE LISTING FOR THE FUC-GM1 ANTIBODY

| SEQ ID NO. | Description | Amino acid sequence |
|---|---|---|
| 1 | Anti-Fucosyl-GM1 | EVQLVESGGGSVQPGESLRLSCVASGFTFSRYKMNWVRQAPG |
| | Heavy Chain | KGLEWVSYISRSGRDIYYADSVKGRFTISRDNAKNSLYLQMNS |
| | Variable Domain | LRDEDTAVYYCAGTVTTYYYDFGMDVWGQGTTVTVSS |
| 2 | Anti-Fucosyl-GM1 | DIQMTQSPSSLSASVGDRVTITCRASQGISSWLAWYQQKPEKAP |
| | Light Chain | KSLIYAASSLQSGVPSRFSGSGSGTDFTLTISSLQPEDFATYYCQ |
| | Variable Domain | QYNSYPPTFGGGTKVEIK |
| 3 | Anti-Fucosy1-GM1 | EVQLVESGGGSVQPGESLRLSCVASGFTFSRYKMNWVRQAPG |
| | Heavy Chain | KGLEWVSYISRSGRDIYYADSVKGRFTISRDNAKNSLYLQMNS |
| | | LRDEDTAVYYCAGTVTTYYYDFGMDVWGQGTTVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKRVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVYTLPPSREEMTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPG |
| 4 | Anti-Fucosyl-GM1 | DIQMTQSPSSLSASVGDRVTITCRASQGISSWLAWYQQKPEKAP |
| | Light Chain | KSLIYAASSLQSGVPSRFSGSGSGTDFTLTISSLQPEDFATYYCQ |
| | | QYNSYPPTFGGGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| 5 | Anti-Fucosyl-GM1 | RYKMN |
| | CDRH1 | |
| 6 | Anti-Fucosyl-GM1 | YISRSGRDIYYADSVK |
| | CDRH2 | |
| 7 | Anti-Fucosyl-GM1 | TVTTYYYDFGMDV |
| | CDRH3 | |
| 8 | Anti-Fucosyl-GM1 | RASQGISSWLA |
| | CDRL1 | |
| 9 | Anti-Fucosyl-GM1 | AASSLQS |
| | CDRL2 | |
| 10 | Anti-Fucosyl-GM1 | QQYNSYPPT |
| | CDRL3 | |
| 11 | Anti-Fucosyl-GM1 | EVQLVESGGGSVQPGESLRLSCVASGFTFSRYKMNWVRQAPG |
| | Heavy Chain (Fc | KGLEWVSYISRSGRDIYYADSVKGRFTISRDNAKNSLYLQMNS |
| | inert) | LRDEDTAVYYCAGTVTTYYYDFGMDVWGQGTTVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKRVEPKSCDKTHTCPPCPAPEAEGAPSVFLFPPKPKDTLMI |
| | | SRTPEVTCVVVDVSHEDPEVKFNWYVDGVEVHNAKTKPREEQ |
| | | YNSTYRVVSVLTVLHQDWLNGKEYKCKVSNKALPAPIEKTISK |
| | | AKGQPREPQVYTLPPSREEMTKNQVSLTCLVKGFYPSDIAVEW |
| | | ESNGQPENNYKTTPPVLDSDGSFFLYSKLTVDKSRWQQGNVFS |
| | | CSVMHEALHNHYTQKSLSLSPG |
| 12 | Anti-Fucosyl-GM1 | DIQMTQSPSSLSASVGDRVTITCRASQGISSWLAWYQQKPEKAP |
| | Light Chain (Fc | KSLIYAASSLQSGVPSRFSGSGSGTDFTLTISSLQPEDFATYYCQ |
| | inert) | QYNSYPPTFGGGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |

### Example 2: Generation of FucGM1 ADC-001 DxD ADC

An anti-FucGM1 antibody (consisting of heavy chain having the amino acid of SEQ ID No: 3 and a light chain having the amino acid sequence of SEQ ID No: 4) was treated with 30 equivalent of tris(2-carboxyethyl)phosphine (TCEP) in PBS, pH 7.4, 2 mM EDTA at 37° C. for 1 hour followed by purification using a ZEBA™ spin column to remove excessive TCEP. The reduced antibodies were eluted with PBS, pH 6.8, 2 mM EDTA and reacted with 10-12 equivalent of maleimide-functionalized payload linker in the same buffer at room temperature for 1 hour. The final product was purified using a HiTrap SP cation exchange chromatography column. The linear salt concentration gradient elution was carried out by changing sodium chloride (NaCl) concentrations from 0 to 1 molar (M). Buffer A was 50 mM sodium acetate pH5, and buffer B 50 mM sodium acetate pH5, 1 M NaCl. The samples were analyzed by LC-MS using an Agilent 1290 Infinity ultra-performance liquid chromatograph (UPLC) system coupled to a 6530 Accurate-Mass Q-TOF mass spectrometer (Agilent, Santa Clara, CA). The analytical column used was a Waters BEH C4 column, 1.7 m, 2.1 mm×50 mm, held at 60° C. The mobile phase consisted of 0.1% formic acid in water (A) and 0.1% formic acid in acetonitrile (B). The system was operated at a flow rate of 200 microliters per minute (μL/min). The gradient condition was as follows: 0-2 min, held at 27% B; 2-9 min, slow ramp from 27% to 37% B; 9-9.5 min, linear ramp from 37% to 90% B; 9.5-12.3 min, held at 90% B. The MS settings were as follows: polarity=positive, capillary voltage=4.2 kV, sample cone=40 V, source offset=15 V, source temperature=140° C., desolvation temperature=325° C. The data acquisition range was 900-3200 m/z. Deconvolution was performed using an Agilent MassHunter Walkup software. Monomer percentage was analyzed by size exclusion chromatography (SEC) using an Agilent 1200 Infinity HPLC. The analytical column used was Zorbax 4 um, GF-250, 9.4×250 mm. The mobile phase consisted of 0.1 M sodium carbonate, 0.2 M methylammonium chloride (MaCl), pH 7.2, 5 mM glycine, 15% acetonitrile (ACN).

### Example 3: Testing Specific Target Binding to FucGM1 of MBN-001 & ADC-001 ADC

Analytical binding studies were carried out by surface plasmon resonance to characterize the binding kinetics of selected anti-FucGM1 antibody MBN-001 and the anti-FucGM1 ADC-001 Dxd (DAR8) ADC to full-length human FucGM1.

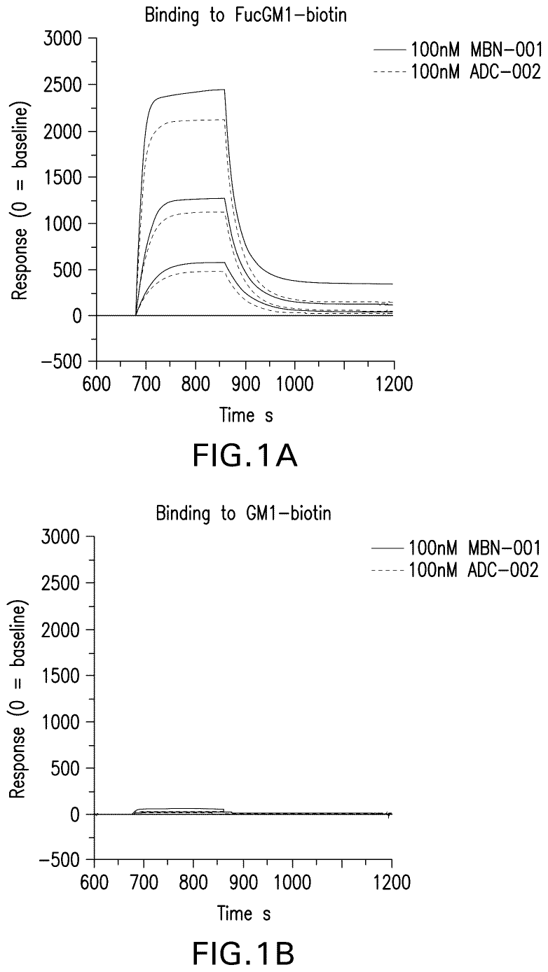

Surface plasmon resonance (SPR) assay analysis was performed to determine the binding of the antibody. The SPR data were collected on a Biacore T200 instrument (Cytiva). The Series S sensor Chip CAP (Cytiva) was docked in the instrument overnight to hydrate in standby mode with running HBS-P+ buffer (0.01 M HEPES pH 7.4, 0.15 M NaCl, 0.005% v/v Surfactant P20; Cytiva). On the day of the experiment, the sensor chip surface was conditioned with a regeneration solution provided in the Biotin CAPture Kit (3 parts of 8 M guanidine-HCl mix with 1 part 1 M NaOH, Cytiva). Biotin CAPture Reagent was captured on the chip according to instructions provided in the kit, followed by capture of biotinylated FucGM1 or GM1 (Elicity1) at a surface density of approximately 100RU. Either MBN-001 or ADC-002 was then applied over captured biotinylated FucGM1 or GM1 (Elicity1) and binding was evaluated at 11.1 nM, 33.3 nM and 100 nM in HBS-P+ running buffer at 37° C. The chip surface was regenerated with 2×2 min pulses of regeneration solution between cycles. The binding levels of MBN-001 and ADC-002 to FucGM1 or GM1 obtained in the avidity SPR assay format described above, were evaluated using the Biaeval software. Reference subtracted sensorgrams of MBN-001 and ADC-002 were overlaid to compare binding profiles. SPR sensorgrams show similar binding of both MBN-001 and ADC-002 to FucGM1 (FIG. 1A), and no significant binding to GM1 (FIG. 1B), indicating the antibody and the ADC are selective to FucGM1.

### Example 4. Testing the ADCC Activity of MBN-001 and ADC-001

ADCC cytotoxicity was tested in multiple SCLC cell lines with a range of FucGM1 cell surface expression. The FucGM1 expression profile of each cell line is shown in Table 4 below.

TABLE 4
Cell Surface Expression (FACS) of
FucGM1 for different cells lines

| | Geometric mean | |
|---|---|---|
| | fluorescence intensity | |
| Cell Line | (gMFI) | Antigen Density |
| NCI-H187 | 16679 | 393,517 |
| DMS-79 | 14540 | 322,059 |
| DMS-53 | 1796 | 15,199 |

For the assay, human primary NK cells were thawed and pre-incubated with 10 ng/ml human IL-2 overnight. The concentration of the target cells was adjusted to a value of 1×10⁶ cells/mL in culture media, and were then labeled with 3 μl DELFIA BADTDA reagent (Perkin Elmer).

After 20 minutes incubation in 37° C., target cells were washed three times with 15 mL culture media. After the final wash, approximately 7500 labeled target cells were mixed with 15000 NK cells. Serial diluted amounts of MBN-001 or ADC-001was added to constitute the designated final concentrations. A maximum release control well was created by treating labeled DMS-79 cell and DELFIA lysis buffer (Perkin Elmer). The cells treated with either MBN-001 or ADC-001 were incubated for three hours. The supernatant for the treated wells was extracted and used to measure Europium signal with a time resolved fluorimetric DELFIA assay kit (Perkin Emmer). The data were normalized to maximum release control as 100% lysis. Data in Table 5 show that ADC-001 preserved ADCC activity compared to MBN-001 after DAR8 DXd conjugation.

TABLE 5
ADCC activity (EC50 nM) data for MBN-001 and ADC-001

| cell type | MBN-001 | ADC-001 |
|---|---|---|
| DMS-79 | 0.0054 | 0.0039 |
| NCI-H187 | 0.1217 | 0.1620 |
| DMS-53 | 0.0494 | 0.0534 |
| Raji (negative | 390 | 1,718 |
| control) | | |

### Example 5: Cytotoxicity Analysis of MBN-001 and ADC-001

This Example assessed the cell viability of different FucGM1 expressing cancer cell lines after treating with MBN-001 and ADC-001 (also referred to as ADC001).

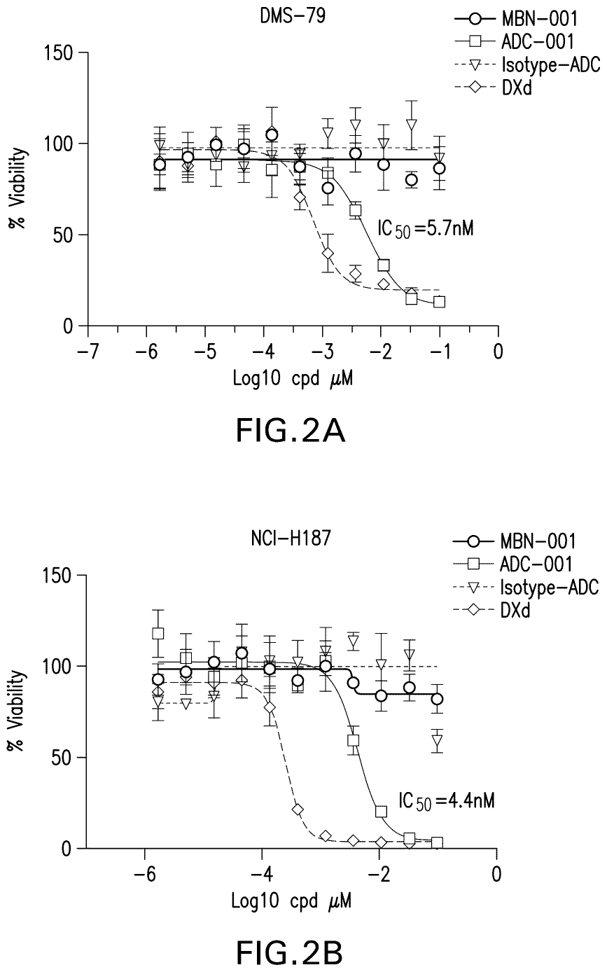

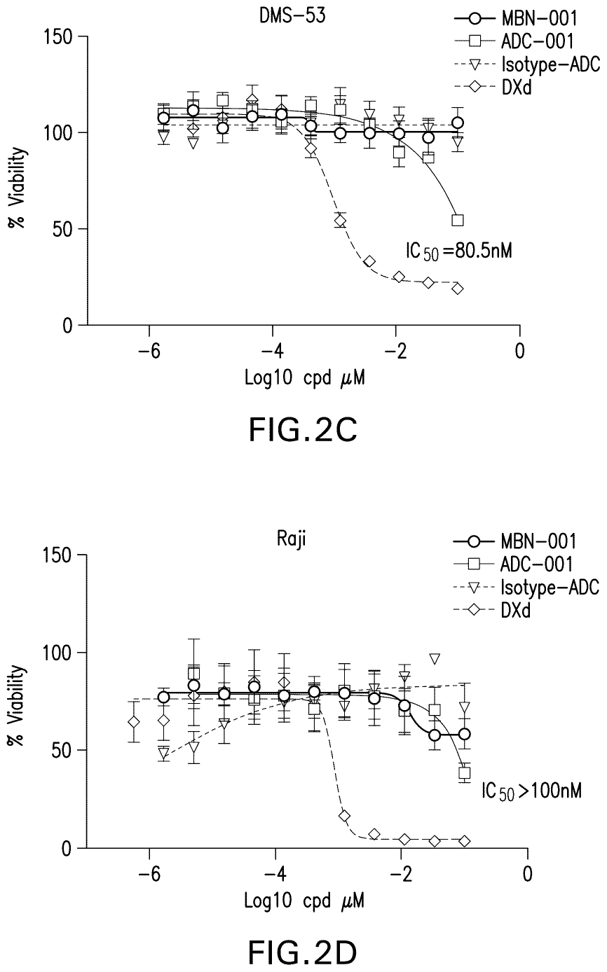

Small cell lung cancer cell lines DMS-79, NCI-H187 and DMS-53, as well as control cell line Raji were maintained in RPMI1640 supplemented with 10% FBS. DMS-79 (FIG. 2A) and NCI-H187 (FIG. 2B) are FucGM1 higher expressing cell line, DMS-53 (FIG. 2C) is FucGM1 lower expressing cell line, and Raji (FIG. 2D) is FucGM1 negative cell line. On day 0, cells were plated (2000 cells/well) in a 384 well plate. Each of MBN-001, ADC-001, isotype-ADC, or DXd alone was serial diluted in culture media, and added to the cells to designated final concentrations shown in FIGS. 2A-2D. On day 5, cell viability was measured by a luminescent cell viability assay using a Cell Titer Glo reagent (Promega). Data were normalized to untreated group as 100% viability. See FIGS. 2A-2D. Data show that treatment with ADC-001 effectively killed the different SCLC cells lines. In fact, treatment with ADC-001 was more effective in reducing cell viability for the different FucGM1-expressing cells lines DMS-79, NCI-H187, and DMS-53 compared to MBN-001, let alone the isotype control or Dxd alone.

### Example 6—Conjugation Reaction with Compound A′ and Anti-FUCGM1 mAb to Yield DAR8 ADCs

This example describes the conjugation of MBN001 to Compound A′ to produce ADC-002. Table 6 lists materials used for the conjugation method.

TABLE 6
Reagents and consumables

| | | Catalog |
|---|---|---|
| Description | Manufacturer | number |
| Zeba Spin desalting columns | Thermo Fisher | 89893 |
| 7 kDa MWCO | Scientific | |
| Amicon Ultra - 1 5 PLTK | Merck Millipore | UFC903024 |
| Ultracel-PL centrifugal | | |
| filters Regenerated | | |
| Cellulose (30 kDa MWCO) | | |
| TCEP HCl, 0.5M, pH 7.0 | Sigma-Aldrich | 646547 |

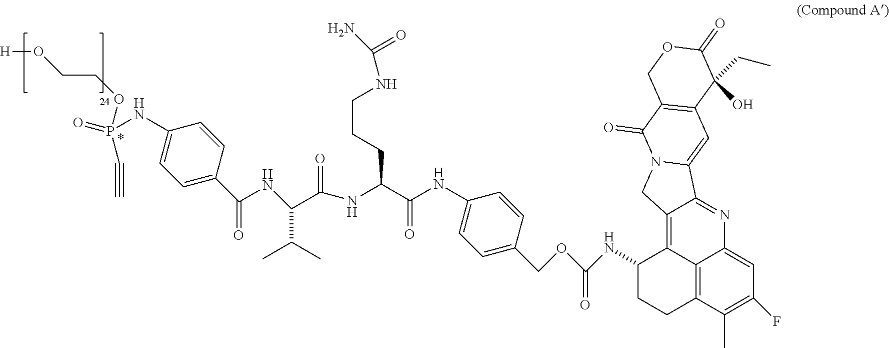

Firstly, each mAb was buffer-exchanged into P5 conjugation buffer (50 mM Tris, 100 mM NaCl, 1 mM EDTA, pH 8.3 at 25° C.) utilizing Zeba Spin desalting columns that were equilibrated with P5 conjugation buffer according to the manufacturer's instructions.

Following buffer exchange the solutions were adjusted to a concentration of 10 mg/ml. Recovery-yields were usually >95%.

For the mAb stock solutions with a concentration below 10 mg/ml, concentration and buffer exchange (diafiltration) were performed using protein concentrator spin columns (Amicon Ultra) according to the manufacturer's instructions. The spin columns were rinsed with P5 conjugation buffer prior to application of the mAb.

After buffer exchange, the mAb concentration was measured with a nanophotometer. P5 conjugation buffer was utilized as the blank. Finally, the mAb concentration was adjusted to 10 mg/ml with P5 conjugation buffer.

Next the mAb was transferred into amber plasticware. The conjugation reaction was performed protected from light. Conjugation with Compound A′ was performed with a molar ratio of 7 eq. TCEP and 10 eq. of Compound A′ per 1 equivalent of mAb. Typically, a 40 mM Compound A′ stock in DMSO and a 10 mM TCEP working solution were used. The 40 mM linker-payload stock solution was thawed. A fresh 10 mM TCEP working solution was prepared by combining 20 μl of 0.5 M TECP-HCl pH 7.0 with 980 μl of P5 conjugation buffer. Both reagents were vortexed prior to use.

The calculated amount of TCEP working solution was added to the solution of the MBN-001 mAb and mixed by gentle swirling. The calculated amount of Compound A′ stock was added immediately afterwards. The mixture was then incubated overnight at 23° C. in amber 50 ml tubes and spun at a speed of 300 rpm.

Conjugation efficiency was evaluated by LC-MS analysis. The conjugate samples were diluted to 1 mg/mL in 100 mM Tris pH 7.5. A volume (20 μl) of sample was reduced by adding 2 μl of 0.5M dithiothreitol (DTT) or TCEP. The samples were analyzed by LC-MS using an Agilent 1290 Infinity UPLC system coupled to a 6530 Accurate-Mass Q-TOF. The analytical column (Waters Inc., BEH C4 column, 1.7 um, 2.1 mm×50 mm) was equilibrated at 60° C. The mobile phase consisted of 0.1% formic acid in water (phase A) and 0.1% formic acid in acetonitrile (phase B). The system was operated at a flow rate of 200 μl/min. The gradient condition was as follows: 0-2 min., held at 27% B; 2-9 min., slow ramp from 27-37% B; 9-9.5 min., linear ramp from 37-90% B; 9.5-12.3 min., held at 90% B. The MS settings were as follows: Polarity=Positive, Capillary Voltage=4.2 kV, Sample Cone=40 V, Source Offset=15 V, Source Temperature=140° C., Desolvation Temperature=325° C. The data acquisition range was 900-3200 m/z. Deconvolution was done using Agilent MassHunter Walkup. If any unconjugated mAb was still present, another 1.4 equivalents of TCEP and 2 equivalents of Compound A′ were added followed by incubation for 2-4 hours. The process yielded DAR8 ADCs.

### Example 7. Testing the In Vitro Properties of ADC-002

This Example assessed the cell viability of different FucGM1 expressing cancer cell lines after treating with MBN-001 and ADC-002 (also referred to as ADC002). Different assays were performed using DMS-79 cells that express FucGM1.

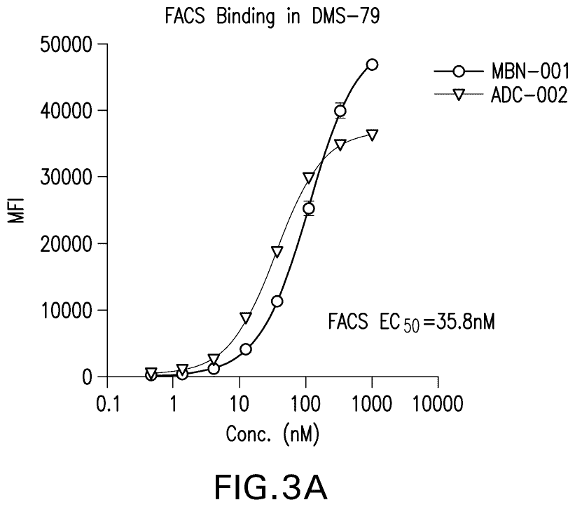

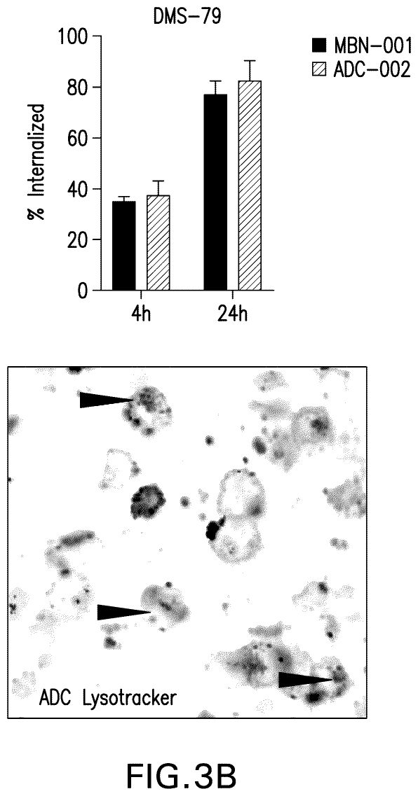

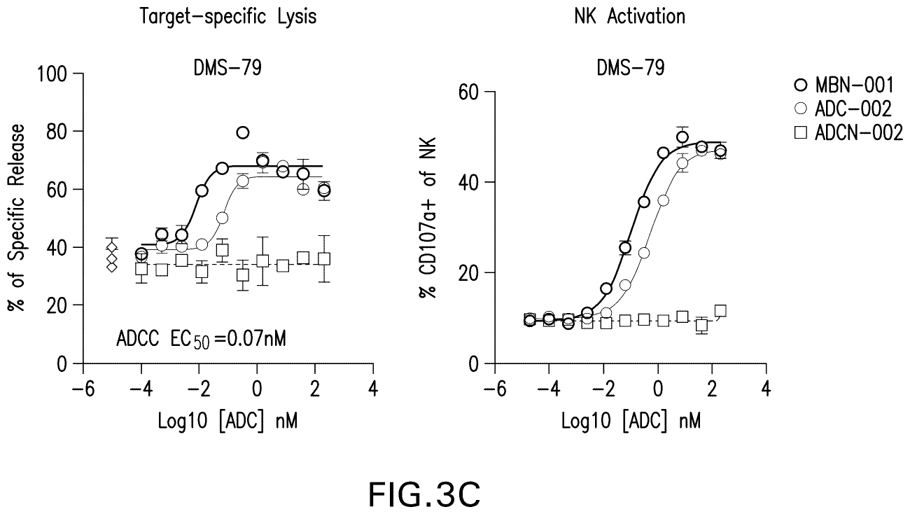

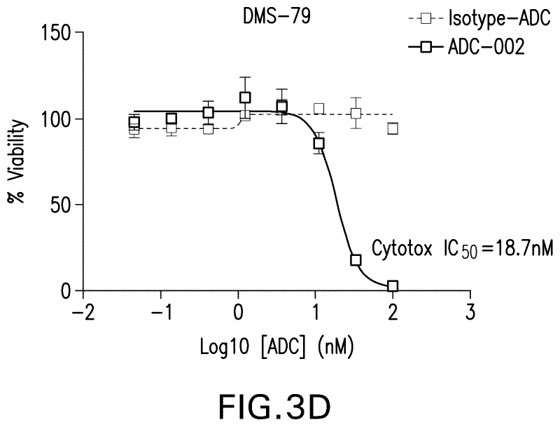

FACS Binding DMS-79 cells were harvested and resuspended in Dulbecco's phosphate-buffered saline (DPBS) solution. Cells were then stained with Live/Dead staining solution in DPBS for 20 min in room temperature. The Live Dead assay staining solution is a mixture of two fluorescent dyes that differentially label live and dead cells. After the Live/Dead staining, cells were incubated with serial diluted MBN-001 or ADC-002 as primary antibody staining, in 4° C. for one hour. Incubated cells were then washed two times with FACS buffer (DPBS with 2% BSA), and stained with 1:2000 diluted goat-anti-human IgG-PE antibody, in 4° C. for 1 hour. Cells were washed twice and FACS binding was analyzed by flow cytometry. Data show that MBN-001 and ADC-002 each bound to the DMS-79 cells (FIG. 3A).

### Lysotracker Colocalization

In another experiment, DMS-79 cells were pre-treated with cell tracker orange dye (ThermoFisher) and plated (10,000 cells/well_in a 384-well PDL-coated Phenoplate microplate (Perkin Elmer). The cells were incubated overnight. On day 1, the cells were incubated (for 1 hour at 4° C.) with Fc Block to block non-specific binding of Fc receptors to antibodies. The cells were washed and were then treated with 50 nM of MBN-001 or ADC-002. The treated cells were incubated for one hour at a temperature of 4° C. The cells were then washed and stained with anti-human IgG-AF488 antibody for 1 hour at a temperature of 4° C. The culture media was added back to the cells and the cells were incubated for approximately 48 hours. The internalization kinetics were measured using an Opera Phenix screening system using a 40× water objective (Perkin Elmer). For the lysotracker colocalization study, the lysotracker dye was added to cells at 24 hours post fixation. Data show that both MBN-001 and ADC-002 were internalized by the FucGM1-expressing cells (FIG. 3B, left and right).

### ADCC Activity

Human primary NK cells were thawed and pre-incubated with 10 ng/ml human IL-2 overnight. The concentration of DMS-79 cells was adjusted to a value of 1×10⁶ cells/mL in culture media. The DMS-79 cells were labeled with 3 μl DELFIA BADTDA reagent (Perkin Elmer). The DELFIA BATDA reagent was useful for labeling of cells in short term cytotoxicity tests utilizing time-resolved fluorometry in the detection.

Cells were incubated in 37° C. for 20 minutes, and washed three times with 15 mL culture media. After the final wash, approximately 7,500 labeled DMS-79 cells were mixed with 15,000 NK cells. Serial diluted amounts of either MBN-001, ADC-002 or ADCN-002 were added to constitute the designated final concentrations. A maximum release control well was created by treating labeled DMS-79 cell and DELFIA lysis buffer (Perkin Elmer). The cells treated with either MBN-001, ADC-002 or ADCN-002 were incubated for three hours. The supernatant for the treated wells was extracted and used to measure Europium signal with a time-resolved fluorimetric DELFIA assay kit (Perkin Emmer). The data were normalized to maximum release control as 100% lysis. The cell pellet for each of the different treated cells was collected for flow cytometry examination of CD107a. Data show that treatment with MBN-001 and ADC-002 induced target-specific lysis and also NK activation in the DMS-79 cells (FIG. 3C, left and FIG. 3C, right).

### Cell Viability Cytotoxic IC50

In a different experiment, DMS-79 cells were harvested on day 0 and prechilled on ice and resuspended in 50 mL culture media. A volume (1 mL) of Matrigel hydrogel (Corning) was added to cell suspension to a final concentration of 2%. The cells were seeded on a 96-well ultralow attachment plate (Costa) at a concentration of 5000 cells/well. The cells were incubated for three days for efficient spheroid formation. On day 3, serial diluted isotype-ADC or ADC-002 were added to the spheroid cultures to the designated final concentrations. On day 10, cells were treated with 100 ul/well CTG 3D assay reagent (Promega), and then incubated (with shaking) at room temperature for 30 minutes. Luminescence of the incubated cells was measured. Cell viability was normalized to untreated wells as 100% viability. Data show that ADC-002 has a cytotoxic IC50 value of 18.7 nM (FIG. 3D).

### Example 8. Testing the In Vivo Anti-Tumor Efficacy of the Anti-FucGM1 ADCs

This Example also analyzed the in vivo anti-tumor efficacy of anti-FucGM1 ADCs ADC-001 and ADC-002 in the cell line-derived xenograft model DMS-79. A number (5×10⁶) DMS-79 cells were prepared in 50% (v/v) Matrigel in PBS and inoculated subcutaneously into the right flank of male C.B-17 SCID mice. Animals with tumor volume between 90 mm³˜244 mm³ were randomized into treatment groups with mean tumor volume of 153 mm³ (N=9/group). Tumor bearing animals were treated with a single intravenous injection of 3 mg/kg isotype control antibody, 3 mg/kg ADC containing an isotype-control antibody conjugated to DXd, or a single intravenous injection (0.3 mg/kg, 1 mg/kg or 3 mg/kg) with ADCs containing MBN-001 described herein: ADC-001 and ADC-002. Tumor length (L) and width (W) were measured with calipers and tumor volumes were calculated using the formulation L*(W\hat{ })/2.

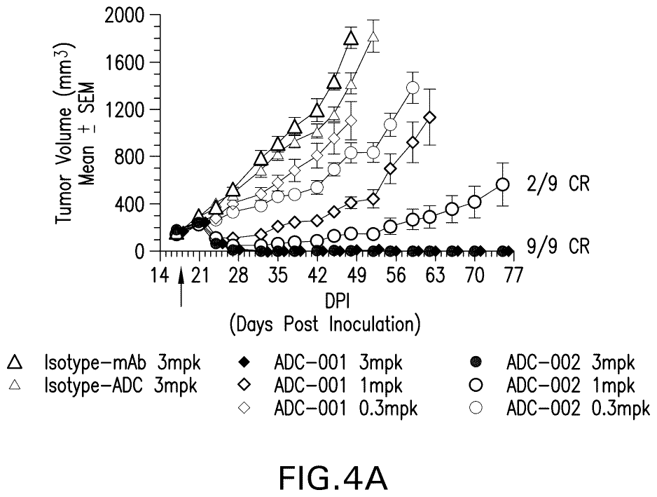

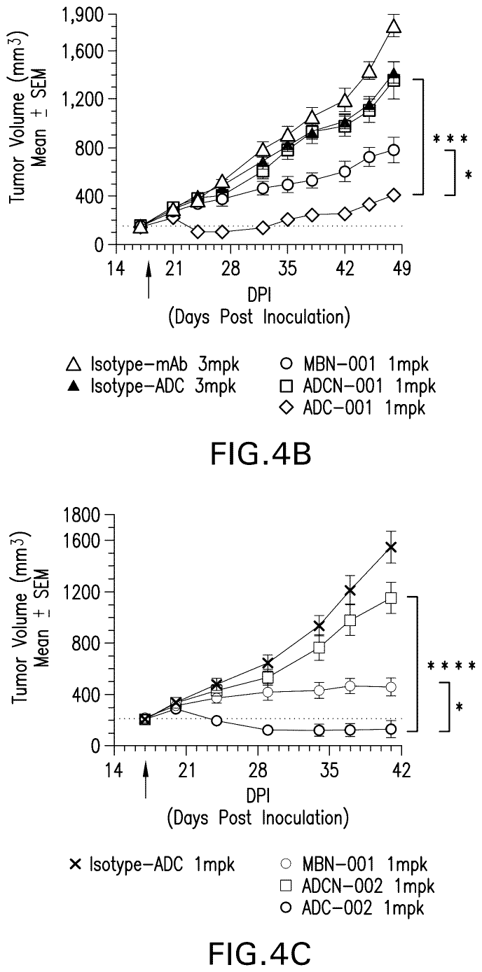

Results for tumor growth of the model treated with ADCs are provided in FIG. 4A. Data show that ADC-002 had dose-dependent response in the DMS-79 model. Superior anti-tumor effect was observed in this in vivo model compared to the isotype control Mab, ADC containing an isotype-control antibody conjugated to DXd, and ADC-001. In fact, data show that treatment with ADC-002 resulted in 99% mean tumor growth inhibition (TGI) 48 days post inoculation (30 days post dose administration) following 1 mg/kg and 100% complete response (CR) following 3 mg/kg. In fact, data show that ADC-002 treatment resulted in superior efficacy compared to ADC-001 at both 0.3 mg/kg and 1 mg/kg dose levels. Furthermore, ADC-002 showed no signs of toxicity for all treatment groups. Additional in vivo efficacy data was obtained for MBN-001, ADCN-001 and ADC-001 at 1 mpk (FIG. 4B) and also MBN-001, ADCN-002 and ADC-002 at 1 mpk (FIG. 4C). ADC-001 had superior efficacy in vivo compared to MBN-001 (ADCC only) or ADCN-001 (inert Fc; ADC consisting of the antibody heavy chain amino sequences of SEQ ID NO: 11 and a light chain amino acid sequence of SEQ ID NO: 12) (FIG. 4B). ADC-002 had superior efficacy in vivo compared to MBN-001 (ADCC only) or ADCN-002 (inert Fc; ADC only) (FIG. 4C). Tumor volume significance was determined by Two-Way ANOVA/Geisser-Greenhouse Correction and Tukey post-hoc test with P-values (*P<0.05; ***P<0.001; ****P<0.0001) recorded at the final measurement.

### Example 9. Testing the Ability of ADC-002 Induce Replicative Stress in Cells

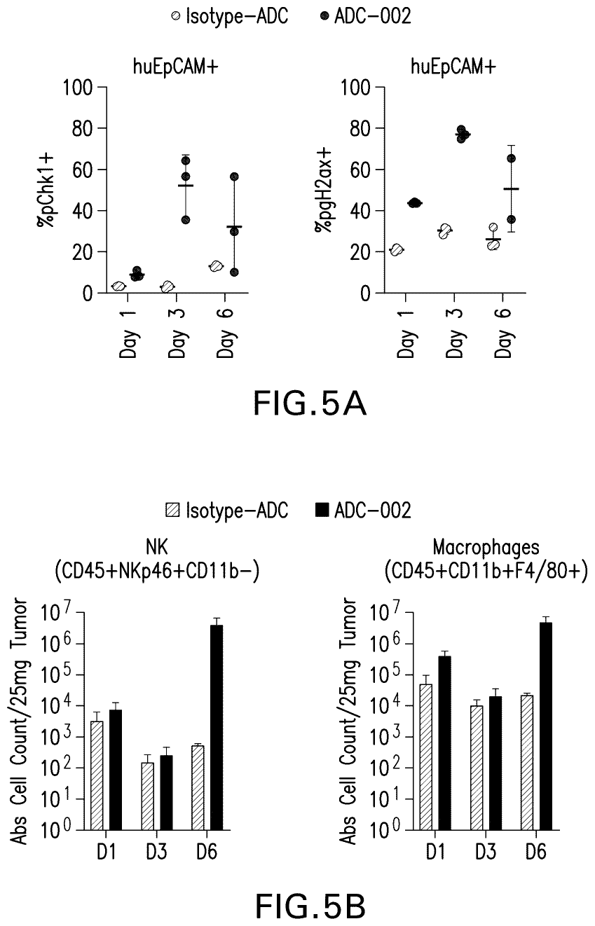

A number (5×10⁶) DMS-79 cells were prepared in 50% (v/v) Matrigel in PBS and inoculated subcutaneously into the right flank of male C.B-17 SCID mice. When the tumor size reached ˜534 mm³, the animals were randomized into treatment groups and intravenously dosed with either 3 mg/kg (mpk) isotype-ADC or 3mpk ADC-002. On day 1, 3, 6 post treatment, the tumors were resected and dissociated into cell suspension using human tumor dissociation kit (Miltenyi). Each cell suspension was adjusted to 100 μp/25 mg initial tumor tissue. A volume (100 μp) cell suspension was then used for staining. The cells were first stained for cell surface targets including anti-human EpCAM, anti-mouse CD45, anti-mouse NKp46 for 20 minutes at room temperature. Next cells were washed and processed with Transcription Factor staining buffer set (BD). Finally, the processed cells were stained for intracellular targets including anti-phospho-CHK1 and anti-phospho-γH2Ax, etc. After washing, the cells were analyzed with flow cytometry. FIG. 5A is a set of graphs showing percentage of phospho-CHK1 positive cells and phospho-γH2Ax positive cells gated within human EpCAM+ cancer cell population, from isotype-ADC or ADC-002 treated tumors. FIG. 5B is a set of graphs showing cell number quantification of NK and macrophage cells in the tumors from isotype-ADC treated tumors or ADC-002 treated tumors. ADC-002 induced DNA damage response biomarker phospho-CHK1 (pCHK1) and phospho-γH2Ax (p-gH2Ax) in tumor cells in vivo, and the response peaked on day 3 (FIG. 5A, left and right). ADC-002 induced NK and macrophage infiltration into the tumor tissue, and the response peaked on day 6 (FIG. 5B, left and right).

### Example 10. Testing the Anti-Tumor Efficacy of the Anti-FucGM1 ADC-002 and B7H3 ADC

This Example analyzed the anti-tumor activity of ADC-002 compared to DS-7300 (a B7-H3-directed antibody conjugated to a topoisomerase I inhibitor payload DXd ADC; Yamato et al., 2022 Mol Cancer Ther 21(4) 635-646).

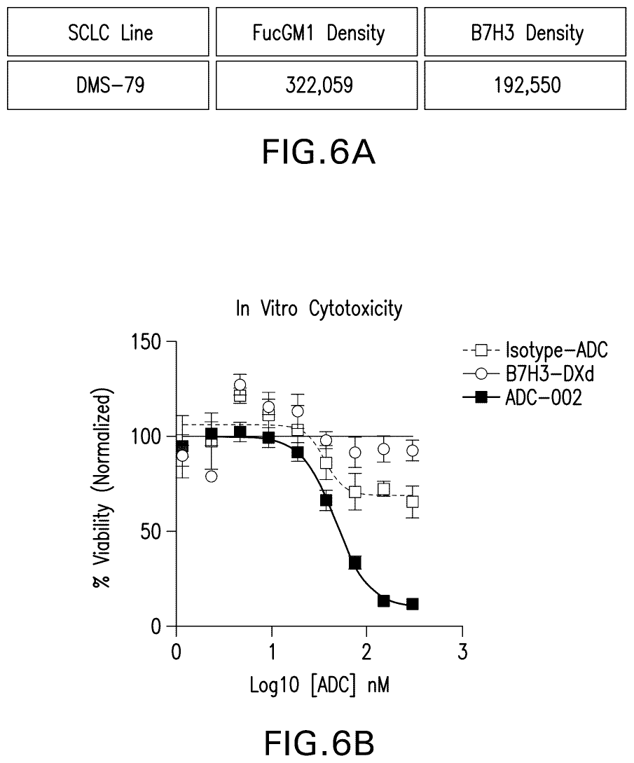

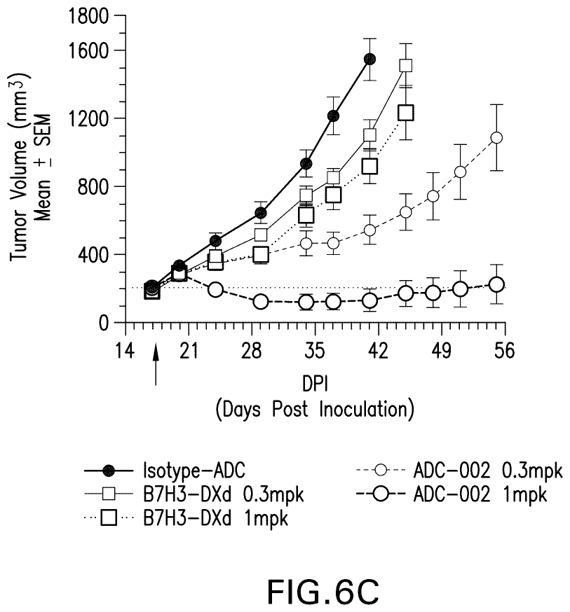

The antigen density of FucGM1 and B7H3 in DMS-79 cells was quantified using a Quantum anti-human IgG kit (Bangs Laboratories). Briefly, DMS-79 cells or quantification beads with defined density of antibody binding moiety were stained with 100 nM AF647 conjugated MBN-001 or B7H3 antibody (antibody from DS-7300), and flow cytometry was used to quantify fluorescence signals. The FucGM1 antigen density and B7H3 antigen density in DMS-79 cells were quantified by interpolating the fluorescence signal using the beads-based standard curve. The densities are shown in FIG. 6A.

The cytotoxicity of ADC-002 was compared to B7H3-DXd ADC DS-7300 in a 3D spheroid cytotoxicity assay. The assay was performed as described in FIG. 3D. Serial titrated isotype-ADC, B7H3-DXd ADC and ADC-002 was added to the cells, and cell viability was measured by 3D CTG. Data show that treatment with the ADC-002 resulted in improved cytotoxic activity in the in vitro assay compared to treatment with the B7H3 Dxd ADC. See FIG. 6B.

In another experiment, scientists analyzed the in vivo anti-tumor efficacy of anti-FucGM1 ADC-002 and DS-7300 in the cell line-derived xenograft model DMS-79. A number (5×10⁶) DMS-79 cells were prepared in 50% (v/v) Matrigel in PBS and inoculated subcutaneously into the right flank of male C.B-17 SCID mice. Animals with tumor volume between 104 mm³˜298 mm³ were randomized into treatment groups with mean tumor volume of 195 mm³ (N=9/group). Tumor-bearing animals were treated with a single intravenous injection of 1 mg/kg ADC containing an isotype-control antibody or with ADCs (0.3 mg/kg or 1 mg/kg): B7H3-DXd and ADC-002. Tumor length (L) and width (W) were measured with calipers and tumor volumes were calculated using the formulation L*(W\hat{ }2)/2.

Results for tumor growth of the in vivo model treated with ADCs are provided in FIG. 6C. Data show that similar to the in vitro cytotoxicity study (FIG. 6B) that ADC-002 had superior anti-tumor effect in these in vivo models compared to the B7H3 targeting-DXd ADC. See FIG. 6C.

Equivalents: Those skilled in the art will recognize or be able to ascertain using no more than routine experimentation, many equivalents of the specific aspects disclosed herein. Such equivalents are intended to be encompassed by the following claims.

1. An antibody drug conjugate (ADC) that binds to fucosyl-GM1 having the formula (II): 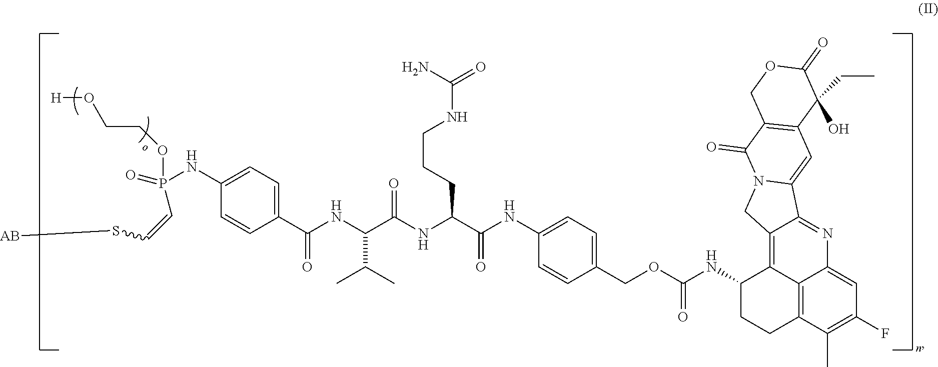
or a pharmaceutically acceptable salt, a stereoisomer or a solvate thereof, wherein: [symbol]
indicates that the configuration of the double bond may be E or Z;
n ranges from 1 to 20; and
AB is an anti-fucosyl-GM1 antibody, or an antigen-binding portion thereof, which comprises a heavy chain variable region (VH) and a light chain variable region (VL), wherein the VH comprises complementarity determining region (CDR) 1 (CDRH1) comprising the amino acid sequence as set forth in SEQ ID NO: 5, CDRH2 comprising the amino acid sequence as set forth in SEQ ID NO: 6, and CDRH3 comprising the amino acid sequence as set forth in SEQ ID NO: 7 and the VL comprises CDRL1 comprising the amino acid sequence as set forth in SEQ ID NO: 8, CDRL2 comprising the amino acid sequence as set forth in SEQ ID NO: 9, and CDRL3 comprising the amino acid sequence as set forth in SEQ ID NO: 10.

2. (canceled)

3. (canceled)

4. (canceled)

5. (canceled)

6. The ADC of claim 1, wherein the heavy chain variable region comprises the sequence set forth in SEQ ID NO: 1.

7. The ADC of claim 1, wherein the light chain variable region comprising the sequence set forth in SEQ ID NO: 2.

8. (canceled)

9. (canceled)

10. (canceled)

11. The ADC of claim 1, wherein the anti-fucosyl-GM1 antibody, or antigen binding portion thereof, comprises a heavy chain comprising the amino acid sequence set forth in SEQ ID NO: 3 or SEQ ID NO: 11.

12. The ADC of claim 1, wherein the anti-fucosyl-GM1 antibody, or antigen-binding portion thereof, comprises a light chain comprising the amino acid sequence set forth in SEQ ID NO: 4 or SEQ ID NO: 12.

13. (canceled)

14. (canceled)

15. The ADC of claim 1, wherein the anti-fucosyl-GM1 antibody, or antigen-binding portion thereof, comprises:
a. a heavy chain comprising the sequence of SEQ ID NO: 3; and
b. a light chain comprising the sequence of SEQ ID NO: 4.

16. The ADC of claim 1, wherein the anti-fucosyl-GM1 antibody, or antigen-binding portion thereof, comprises:
a. a heavy chain comprising the amino acid sequence set forth in SEQ ID NO: 11; and
b. a light chain comprising the amino acid sequence set forth in SEQ ID NO: 12.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The ADC of claim 1,
wherein n ranges from about 4 to 9; o is an integer ranging from 10 to 30; AB is an anti-FucGM1 antibody comprising a VH and a VL, which VH comprises the amino acid sequence set forth in SEQ ID NO: 1 and the VL comprises the amino acid sequence set forth in SEQ ID NO: 2, respectively, wherein the anti-fucosyl-GM1 antibody is non-fucosylated.

36. An antibody drug conjugate (ADC) having the formula (IV): 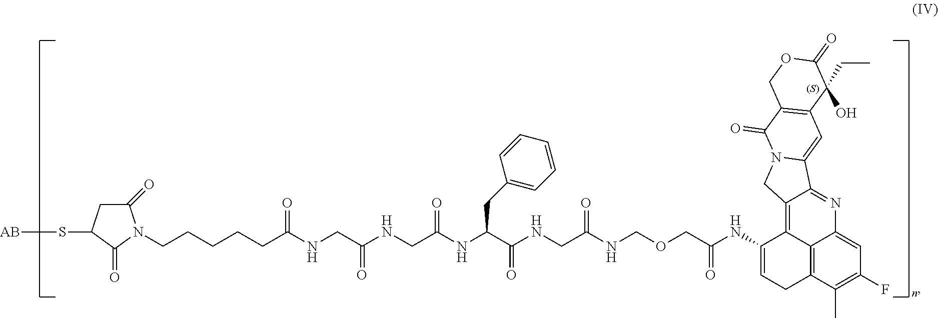
or a pharmaceutically acceptable salt, a stereoisomer or a solvate thereof; wherein n ranges from about 4 to 9; AB is an anti-FucGM1 antibody or antigen binding portion thereof comprising a VH and a VL, which VH comprises the amino acid sequence set forth in SEQ ID NO: 1 and the VL comprises the amino acid sequence set forth in SEQ ID NO: 2, respectively.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. The ADC of claim 1, wherein o is 22, 23, 24, 25, or 26.

55. The ADC of claim 1, wherein n is an integer ranging from 6 to 8.

56. (canceled)

57. (canceled)

58. (canceled)

59. (canceled)

60. A kit comprising the ADC claim 1 and instructions for use.

61. An in vitro method of killing cells expressing FucGM1 comprising contacting a sample comprising the cells with the ADC of claim 1.

62. A method of treating cancer associated with expression of FucGM1 comprising administering to a subject in need thereof the ADC of claim 1.

63. The method of claim 62, wherein the cancer comprises colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, myelodysplastic syndromes, or any combination thereof.

64. The method of claim 63, wherein the lung cancer is small cell lung cancer (SCLC).

65. (canceled)

66. (canceled)

67. (canceled)

68. (canceled)

69. (canceled)

70. A method of producing the ADC of claim 1 comprising conjugating
a thiol containing compound, AB—(SH)ₙ with Compound A′: 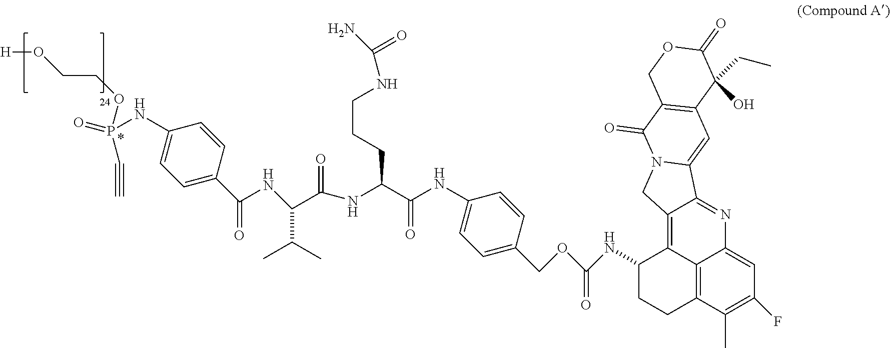
wherein AB is the anti-fucosyl-GM1 antibody or antigen binding portion thereof, and n is 8.

71. An ADC having the formula: 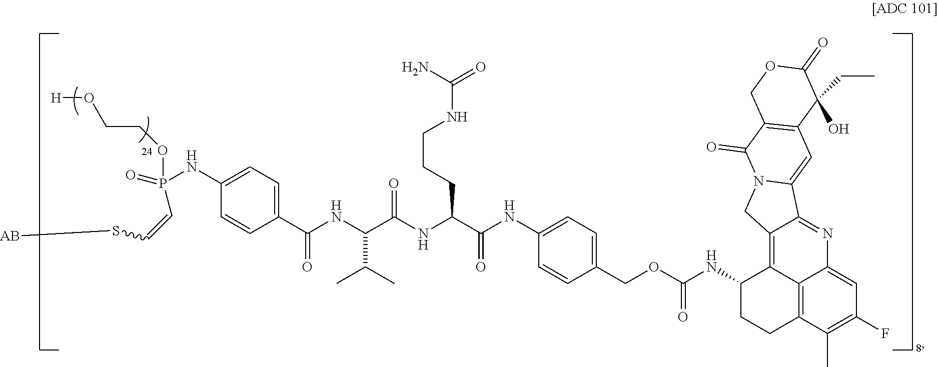
wherein [symbol] indicates that the configuration of the double bond is E or Z;
wherein AB is an anti-FucGM1 antibody comprising a heavy chain variable domain (VH) and a light chain variable domain (VL), which VH comprises the amino acid sequence set forth in SEQ ID NO: 1 and the VL comprises the amino acid sequence set forth in SEQ ID NO: 2, respectively, wherein the anti-fucosyl-GM1 antibody is non-fucosylated.

72. An ADC having the formula: 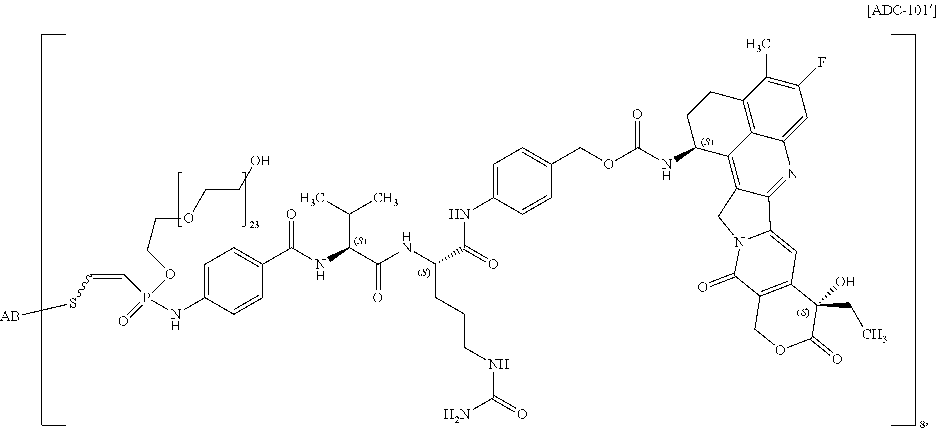
wherein [symbol] indicates that the configuration of the double bond is E or Z;
wherein AB is an anti-FucGM1 antibody comprising a heavy chain comprising the amino acid sequence set forth in SEQ ID NO: 3; and a light chain comprising the amino acid sequence set forth in SEQ ID NO: 4, respectively.

73. (canceled)

74. (canceled)

75. The ADC of claim 71, wherein the anti-fucosyl-GM1 antibody consists of two heavy chains and two light chains, wherein each heavy chain comprising the amino acid sequence of set forth in SEQ ID NO: 3; and each light chain comprising the amino acid sequence set forth in SEQ ID NO: 4.

76. (canceled)

77. (canceled)

78. (canceled)

79. A pharmaceutical composition comprising the ADC of claim 71, and a pharmaceutically acceptable carrier.

80. A method of treating cancer associated with expression of FucGM1 comprising administering the ADC of claim 71.

81. The method of claim 80, wherein the cancer comprises colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, myelodysplastic syndromes, or any combination thereof.

82. The method of claim 81, wherein the lung cancer is small cell lung cancer (SCLC).

83. The method of claim 80, further comprising administering one or more additional therapies.

84. The method of claim 83, wherein the one or more additional therapies comprises radiation therapy, chemotherapy, immune checkpoint inhibitor therapy, CAR-T therapy, immunosuppressive therapy, immunostimulatory therapy, cell therapy, a therapeutic agent, or any combination thereof.

85. (canceled)

86. (canceled)

87. (canceled)

88. A method of treating cancer associated with expression of FucGM1 comprising administering the ADC of claim 72.

89. A pharmaceutical composition comprising the ADC of claim 72, and a pharmaceutically acceptable carrier.

90. A method of treating cancer associated with expression of FucGM1 comprising administering the ADC of claim 75.

91. A pharmaceutical composition comprising the ADC of claim 75, and a pharmaceutically acceptable carrier.


SEQUENCE LISTING (12 sequences)

SEQ ID NO: 1 - protein, 122 aa, Homo sapiens
EVQLVESGGGSVQPGESLRLSCVASGFTFSRYKMNWVRQAPGKGLEWVSYISRSGRDIYYADSVKGRFTISRDNAKNSLYLQMNSLRDEDTAVYYCAGTVTTYYYDFGMDVWGQGTTVTVSS

SEQ ID NO: 2 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 3 - protein, 451 aa, Homo sapiens
EVQLVESGGGSVQPGESLRLSCVASGFTFSRYKMNWVRQAPGKGLEWVSYISRSGRDIYYADSVKGRFTISRDNAKNSLYLQMNSLRDEDTAVYYCAGTVTTYYYDFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Homo sapiens
RYKMN

SEQ ID NO: 6 - protein, 16 aa, Homo sapiens
YISRSGRDIYYADSVK

SEQ ID NO: 7 - protein, 13 aa, Homo sapiens
TVTTYYYDFGMDV

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYNSYPPT

SEQ ID NO: 11 - protein, 451 aa, Homo sapiens
EVQLVESGGGSVQPGESLRLSCVASGFTFSRYKMNWVRQAPGKGLEWVSYISRSGRDIYYADSVKGRFTISRDNAKNSLYLQMNSLRDEDTAVYYCAGTVTTYYYDFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, Homo sapiens; identical to SEQ ID NO: 4